(19) United States
(12) Patent Application Publication
(10) Pub. No.: US 2007/0193632 A1
(43) Pub. Date: Aug. 23, 2007

(54) Artificial heart valve and rotary pressure porting mechanisms

(76) Inventor: Jianchao Shu, Winston, GA (US)

Correspondence Address: Jianchao Shu, 3719 Falls Trail, Winston, GA, 30187, US

(21) Appl. No.: 11/707,345

(22) Filed: Feb. 17, 2007

Related U.S. Application Data
(60) Provisional application No. 60/775,221, filed on Feb. 21, 2006.

(51) Int. Cl. F16K 15/00; A61F 2/24

(52) U.S. Cl. 137/512; 623/2.21; 623/2.28; 623/2.24; 623/2.26

(57) ABSTRACT

This invention provides a simple, rotary, reliable, compact, anticoagulant, artificial or mechanical heart valve based on two rotary pressure porting mechanisms. This valve provides one center flow stream without any obstruction which only human heart valve can provide so far. It employs about ⅕ of the energy that the conventional mechanical heart valve consumes. This mechanical heart valve includes three basic configurations with one leaflet, two and three leaflets for various human heart valve replacement. This valve has the most reliable designs over all existing mechanical heart valves. The reliable features includes less moving part, dual redundant hinge—actuation system and inclusive designs; either loosing proof or falling out proof. Finally most importantly, the invention is provided with an unique anticoagulant mechanisms which include mechanical and medical methods to clean up clots and prolong life of this valve up to 20-40 years. Above all this valve meets and exceed most performance requirements of human heart valve. These rotary pressure porting mechanisms have vast applications and many advantages over conventional linear or rotary pressure porting mechanisms and applied to check valves, pressure relief valves and pressure regulators or their combinations. They can be employed for many fields including medical devices, aerospace industries, automotives, process controls, food process, chemical plants, oil and refinery, HVAC and others.

### CROSS REFERENCE TO RELATED APPLICATION

Provisional Patent Application Ser. No. 60/775,221 filed Feb. 21, 20006

### FEDERALLY SPONSORED RESEARCH

Not Applicable

### SEQUENCE LISTING OR PROGRAM

Not Applicable

### BACKGROUND

1. Field of Invention

This invention relates to novel rotary pressure porting mechanisms, more particularly to a simple, reliable, compact, anticoagulant artificial or mechanical heart valve based on the rotary pressure porting mechanisms. This valve only employs about ⅕ of the energy which conventional mechanical heart valves consume. This valve meets and exceeds most performance requirements of human heart valve including anticoagulant. Finally these rotary pressure porting mechanisms have vast applications and many advantages over conventional linear or rotary pressure porting mechanisms.

2. Description of Prior Art

The first mechanical heart valve was developed about 50 years ago, it was based on a conventional check valve mechanism or linear pressure porting mechanism which opens with forward flow and closes against reverse flow. The conventional mechanical heart valves evolved from a caged ball check valve, the title disc valve, bileaflets swing heart valve to tri-leaflet heart valve, but the basic mechanisms do not change, they are operated with linear mechanism or conventional hinge mechanism. Furthermore the conventional mechanical heart valves have inherently relatively high inertia, high energy consumption, high closing impact force and high leakage. In addition, most conventional mechanical heart valves based on the conventional hinge mechanism also have backflow (regurgitation) and do not close coordinately with heart stroke cycle, as a result the regurgitation flow takes place. The regurgitation flow or backflow not only can cause damage on the valve and blood cells, but also reduce heart pumping efficiency. Finally, the coagulation problem is still unsolved, the patients with the mechanical heart valves have to take anticoagulant drugs for life. Above all, the mechanical heart valves are never designed or considered to meet or exceed the performance requirements of the native heart valve.

In order to overcome the disadvantages of the conventional mechanical heart valve and conventional pressure porting mechanisms, many efforts have been made in the prior arts. There are many approaches to improve the conventional pressure porting mechanisms and mechanical heart valves in the prior arts, but those approaches are not systematic and sometime work against each other within a limited scope they are grouped to four approaches, especially last twenty years, most prior arts are stressed in improving bileaflets heart valve.

The first approach is to improve the hemodynamics and reduce energy loss with one leaflet or valve member heart valve. The first successful mechanical heart valve was a caged ball valve, pioneered by Starr and Edwards, based on the ball valve of U.S. Pat. No. 19,323 to Williams (1858), but the caged ball blocks the center flow stream, and damages the blood cells. Soon the hemodynamic concept of the single tilting disk valve was introduced for improvement over the caged ball valve because it reduces energy loss, and therefore largely replaced the caged ball implant. U.S. Pat. No. 3,546,711 to Bokros ( 1968) discloses a single tilting disk heart valve with journal hinges set away from the orifice wall. U.S. Pat. No. 3,835,475 to Child (1974) discloses a free-floating disk that is constrained by projections. U.S. Pat. No. 4,306,319 to Kaster ( 1981) discloses a tilting disk heart valve with an oval shaped disk and orifice. In this valve, the disk is hinged with an axis of rotation across the largest dimension of the orifice. The tilting disk heart valves have improved flow characteristics over the caged ball valves, but still partially obstruct the central flow of blood while open.

The second approach is to improve performance and reduce energy loss with bi leaflet mechanical heart valve in three following aspects (1) improvement of hinge mechanism. U.S. Pat. No. 4,276,658 to Hanson et al. (1981) shows the most popular hinge design in mechanical heart valves on the market, the combination of the hinge and stopper reduces blood stagnation, but such arrangement reduces concentricity between valve internal surface and leaflets and dynamic performance, as a result, the leakage and vibration increase, and the flat cross section of hinge pin reduces the strength of leaflet and increase stress concentration (2) reduction of leakage. Most conventional bileaflets hear valves have two seals between the valve body and the leaflets, and between the leaflets, those seals are all face to face seal which constitutes a seal only at a relative position between a valve member and a valve body, by nature, no leaflet is made perfectly, so the valve either has a good seal between the bileaflets, a bad seal between the valve body and the leaflets or vise verse. The first approach is to eliminate a seal between leaflets and is widely adopted in the industrial double disc swing checks, but it blocks the center flow stream as shown in U.S. Pat. No. 3,903,548 to Nakib ( 1975). The second approach is to improve the seal surface profile as shown in U.S. Pat. No. 5,405,382 to Kukolmikov et al ( 1995), the seal is provided between a valve and a leaflet with an spherical internal surface of the valve and a spherical surface on the leaflet edge, in fact this seal is still a conventional face to face seal with five problems, first there are two large flat areas connected with the spherical surface, if the flat area is a dominated position factor, then the spherical surface can not seal well and vise verse, second the joint area between the spherical and flat area can cause leakage and jam the leaflets, third the seal surface on the leaflet depends on the thickness of leaflet, in some cases, the thickness is too small to provide a seal, fourth the leaflets block the flow stream and create three flow streams just like conventional bileaflets valve when the valve is at open position, fifth without a pivot pin as a pivoting center, the closing force can be very high in some points around the seal surface and cause chatter and vibration. Finally there are other two approaches which are similar, one is to have a flexible valve body to compensate any geometric error of a leaflet as shown in U.S. Pat. No. 5,397,348 to Campbell et al. (1995), other is to make flexible leaflets to compensate any geometric error of a leaflet as shown in U.S. Pat. No. 6,139,575 to Shu et al Campbell et al. (2000), two approaches may work if mechanical heart valve works less frequently or in a static condition, but in a dynamic condition, opening or closing, the flexible valve body or leaflets became a spring in a valve dynamic operation system to store and release the leaflets kinetic energy rather than as a dumping device to dissipate the energy, as a result, the coefficients of restitution for the valve body and the leaflet become closed to one, those approaches can cause even more leakage, flutter and chatter (3) improvement of hemodynamic performance by changing shape of leaflets or valve internal profiles. The conventional leaflets are divided to two uneven sections by pivot axis, one larger sections is rotated clockwise and other is anticlockwise or vise verse, the difference between two sections under fluid pressure generates a torque to operate leaflets, so the hinge mechanism not only wastes flow energy and leaflet spaces but also has two opposite flow streams around the leaflet and cause backflow when the leaflet moves, the hinge problem is not consciously recognized. U.S. Pat. No. 4,274,437 to Watts. (1981) shows two conical leaflets engaged with two partial spherical surfaces on a valve body, it generates one center flow stream only when the valve is at fully open position, but the hinge mechanism still is conventional, the lager section is against to smaller section, so there are the backflow and multiple streams when the valve is approached to closed or open positions. U.S. Pat. No. 5,376,111 to Bokros et al (1994) shows the same type of leaflets with a body having a cylindrical internal surface, the blood flow is blocked by the two conical leaflets and become three flow streams through the valve when the valve is either at open position or approached to open, the backflow still exist. So in order to solve the backflow problem which is inherent with the conventional hinge mechanism, a new solution was presented by adding a backflow preventing device as shown in U.S. Pat. No. 6,638,303 to Louis A. Campbell (2003), a spring or magnetic blocks was added to increase closed speed for preventing reverse or back flow, but there are three problems, first with conventional hinge mechanism, the backflow caused by the small section on the leaflet still exist, the improvement is very limited, second, the spring or magnetic force will increase closing impact force, third the spring installed in an open manner can fall into blood flow stream if broken, it is not accepted by the medical community, on other hand the magnets block has a manufacturing problem as well as application limitation, to bond the magnetic blocks to carbon is not an easy job, moreover a patient who has a pace maker can not have a mechanical heart valve with the magnetic block, which will disturb the pace maker.

The third approach is to improve hemodynamics and reduce energy loss with tripleaflet mechanical heart valve. The tripleaflet mechanical heart valve was first developed, because it mostly resembles nature heart valve, but it never have successful clinical trials due to complexity of the structure. U.S. Pat. No. 5,843,183 to Bokros (1998) discloses an improved tripleaflet mechanical heart valve with hinge devices on the valve with stopper blocks. U.S. Pat. No. 6,896,700 to Lu et al (2005) shows an improved tripleaflet mechanical heart valve with hinge pin inserted in the valve body, although the leaflets can free move, but they are not secured and can fall out under blood flow, the profile of hinge and stopper are complicated and expensive to make, finally no clinical trials proves any tripleaflet mechanical heart valve as an alternative to bileaflets mechanical heart valve.

The four approach is to improve the pressure porting mechanisms which are basic mechanism behind all check valve and pressure control valves. The conventional pressure porting mechanisms comprises a linear and a hinge mechanisms. The linear pressure porting mechanism is shows in U.S. Pat. No. 19,323 to Williams (1858) and U.S. Pat. No. 544,376 to Porter in 1895. The hinge mechanism was invented by W. Brovo (1917) in U.S. Pat. No. 1,238,878. Since then many improvements were made, but no fundamental changes have been made in two pressure porting mechanisms, the main disadvantages of those two mechanisms still remain (1) center flow stream obstruction or flow stream obstruction (2) high impact closing force (3) size dependency. U.S. Pat. No. 6,953,026 to Yu et al. (2005) shows a pressure regulator with an improved shape of the closure member in a fuel delivery system, but the center flow stream is blocked, as a result it increases the response time and pressure loss and damages the valve. U.S. Pat. No. 5,794,652 to Mizusawa (1988) shows an improvement of check valve used in a fuel delivery system, it reduce central flow obstruction, but the valve member still block the flow. U.S. Pat. No. 3,007,488 to Wheeler ( 1961) discloses an improvement of two plates check valve, but the center flow stream is blocked, U.S. Pat. No. 5,078,177 to Tartaglia et al ( 1991) shows an improved relief valves with a cavity to provide a soft closing, but because of the nature of face to face seal with a biased spring, the soft seal is very difficult to achieve with only dumping mechanism which is a viscous friction between the gas fluid and the valve member in the cavity, thus the performance can be unpredictive. U.S. Pat. No. 6,749,592 to Lard ( 2004) and U.S. Pat. No. 6,770,062 to Phung et al (2004) shows a pressure regulator and a check valve in a chest drainage system, which are based on conventional linear porting mechanism, they block the center flow stream. The pressure regulator is not compact for mobile applications and both are complicated and expensive to produce for a disposable device, above all the closing forces still cause noise in both valves. U.S. Pat. No. 6,588,428 to Shikani et al (2003) shows a speaking valve, which is based on the conventional linear porting mechanism, it blocks the center flow stream and effect the speech quality and distort the patient true voice and is susceptible to air duct. U.S. Pat. No. 4,758,224 to Siposs (1988) and U.S. Pat. No. 6,053,896 to Wilson et al (2000) disclose a combination of check valve and relief valve in a drainage system for the heart surgery operation, but the basic disadvantages of this type of drainage system is that it can not sustain a negative pressure in the inlet of check valve due to flexible, soft leaflet of duckbill check valve, slow response to a pressure change and no pressure setting adjusting device in the relief valve, as a result, the relief valve is required to control the pressure within both the high and lower limits and a very complicated control system for the pump in the suction side. U.S. Pat. No. 6,050,081 to Jansen et al (2000) ) shows a two-way check valve, which is based on the conventional linear porting mechanism, it blocks the center flow stream and cause high pressure loss and coking.

In short, those prior arts provide no single valve meets the performances of human heart valve and or addresses the root of causes of all the disadvantages of conventional mechanical heart valve and conventional pressure porting mechanisms in a systematic manner. The high energy exchange between the valve and fluid through the valve is a root of causes of all the disadvantages and blood clot. When two solid parts have a relative motion in the valve, some energy exchange takes place as a restoring form like spring or potential energy, some energy exchange takes place as a dissipating form (creating a third part or as other form of energy, like heat) through friction, wearing like hinge and the body, when the fluid and solid part have a relative motion, some of energy exchange takes place between kinetic energy and potential energy, some energy exchange takes place through a dissipating form (creating a third part or other form of energy like heat), an erosion which is that the fluid takes away solid material, or buildup (coagulation) which is that the solid part takes away some part of the fluid, finally the heat exchange, the temperature difference between the mechanical heart valve and the surrounding tissue and blood causes energy exchange, even the chemical reaction or biologic reaction is explained by the energy exchange, without energy exchange, nothing happens. So the performance of the mechanical heart valve is depended on the level of energy exchange in three aspects (1) pressure porting mechanism which is an interaction device between the valve and fluid (2) property of the valve materials (3) property of the fluid, so the less energy exchange takes place in the valve, the less damage of blood cell, less coagulation the mechanical heart has, but what is a better mechanical heart valve or a pressure porting mechanism should be, many arguments and research, investigation were published, but they are sometime contradiction and confusion, there is no convincing theory or supporting data to explain how blood fluid mechanism through the heart valve, fortunately, we have a good example, our human heart valve, the following is our human hear valve's strengths
1 One central flow stream
2 No obstructive in the center flow stream
3 No closed force between valve body and valve leaflet
4 Soft closing between the leaflets
5 Self absorb clot materials and self repair mechanism
6 Leaflets internal surface contact flow stream
7 Valve body and leaflet as a integral part
8 Seal among the leaflets
9 Shorter travel between open and closed positions
10 No hinge mechanism
11 Longevity
   And weakness
   1. Center jet flow leakage when the three leaflets are closed
   2. Higher energy consumption to rotate the leaflet with the large normal surface of leaflet to the flow stream.
   3. Susceptible to disease

Those strengths and weakness are an ultimate standards to judge wither or not a mechanical heart valve qualified as a good alternative to nature heart valve or a better pressure porting mechanism.

So the medical device industry and medical community have long sought a means of improving the performance of mechanical heart valve having most of native heart valve strengths and with the few weakness, increasing reliability and anticoagulant ability and life expediency of the valve.

In conclusion, insofar as I am aware, no mechanical heart valve was formerly developed to have higher performances with a anticoagulant structure, less parts, high durability, reliability, easy manufacturing at low cost. Above all, such a valve is as good as or even better than the native heart valve in some aspects.

### SUMMARY

This invention provides a highly reliable, anticoagulant compact, efficient, mechanical heart valve. The mechanical heart valve is based on two novel rotary pressure porting mechanisms to control a flow and provide one constant central flow stream when the valve is approached to opening or closed positions. One is a hybrid functions pressure porting mechanism, it has two sections on a valve member, a spherical shell balanced section and an actuation section, only the actuation section under pressure difference generates open and closed torques. The other is an offset pressure porting mechanism which has a spherical shell valve member with an eccentric pivot axis, so the operating torque is generated by an offset and surface area of the spherical shell, so the operating torque for those two porting mechanisms can be carefully designed to balance the closing speed and impact force. This invention also provides multiple novel hinge joint mechanisms between the valve body and the valve member with less blood cell damage and less blood stagnation and in smooth concentric operation. An unique soft closing concept and mechanism is introduced in this invention, the closing mechanism comprises two stages, first stage is to impact/lock to absorb most leaflet kinetic energy to reduce coefficients of restitution to closed zero, second stage is to contact/seal, so the flutter, chatter and cavitations can be prevented or minimized even when the valve member still oscillate around the closed position. Finally the valve member acts like a cutter to remove any buildup or coagulation on the valve porting surfaces when the valve is closing or opening, while the spherical surfaces on the valve body or leaflet can be coated with anticoagulant drug and release the drug into blood fluid when the valve is closing or opening.

The valves can be constructed with one valve member, two valve members, three valve members in series or parallel or mixed manners. The two rotary pressure porting mechanisms can be applied separately or together and can be used with the conventional linear porting mechanism for other applications other than the mechanical heart valve. A spring or automatic control mechanisms may be equipped for check valve, relief valve or pressure regulator or multiple function valves or control system. Accordingly, besides objects and advantages of the present invention described in the above patent, several objects and advantages of the present invention are:
(a) To provide a valve with one constant central flow stream when valve is opening or closing, so the valve has the most efficient flow pattern with less energy loss and fluid composition damage
(b) To provide a valve without any obstructive in a flow stream, so the valve has most effect flow pattern with less energy loss and blood cell damage.
(c) To provide a valve without closed forces between a valve body and a valve member when the valve is open or closed, so fluid composition will not be damaged and or clot or buildup will not grow in the contact areas, the valve can be constructed much compactly, the noise and vibration causes by the closing force can eliminated.
(d) To provide a valve with a soft closed force, so the fluid composition damage, clot or buildup can be reduced, as a result a seal in the valve can be improved and the valve has long life expediency.
(e) To provide a valve with self clearing mechanism, so in case the clot or buildup grows on the valve critical areas, the valve can clear up the clot and keep proper function without additional anticoagulants or clearing agent.
(f) To provide a valve with only valve member internal surface contact flow stream, so the contact area between valve and flow fluid is reduced, so the flow pressures loss is reduce, so do the damage of fluid composition around the boundary layer and the clot.
(g) To provide a valve with a lock mechanism, so when the valve is approached to a closed position, valve members or valve members and a valve body in the valve can lock together to dissipate the kinetic energy of the valve members, the chatter and flutter can be reduced and eliminated, so do the cavitations, leakage and noise and wearing.
(h) To provide a valve with one face to face seal for each valve member, so such a valve can have good seals between the valve members and the valve member and a valve body at same time in the valve.
(i) To provide a valve with multiple configuration choices, so each valve can be designed to meet each different application requirement.
(j) To provide a valve with simple, robust, reliable, safe hinge mechanisms. So the valve with the hinge mechanism has less damage of fluid composition, fluid stagnation. While the hinge devices provided with a smooth rotation and a better dynamic performance are easily manufactured and assembled at lower cost.
(k) To provide a valve with optimal rotating angles, porting characteristics and closing time to reduce the energy consumption, turbulence and fluid stagnation
(l) To provide a mechanical heart valve with an anticoagulant mechanism to constantly clear up the valve porting surfaces and release the anticoagulant drug, so the patient does not need to take anticoagulant drug for life after the mechanical heart is implanted.
(m) To provide a mechanical heart valve with long life expediency. So the valve has long life expediency up to 20-40 years without inferior performances or additional operation.
(n) To provide novel rotary pressure porting mechanisms, so the valves based on those mechanisms are durable, robust and reliable, versatile and efficient with less weight, parts and cost.
(o) To provide rotary pressure porting mechanisms, so valves with the porting mechanisms have quick response and maximum flow rate with less energy loss.
(p) To provide a fully balanced rotary pressure porting mechanisms for a mechanical heart valve, so the valve is not susceptible to vibration, position or motion changes of a patient or the environment where a patient stays.
(q) To provide hemodynamic leaflet for a mechanical heart valve, so the valve can be used for all patients without backflow.
(r) To provide a mechanical heart valve with highly reliable, inherently redundant, intrinsically safe control means, so the valve can be used for all patients for 20-40 years period.
(s) To provide a mechanical heart valve with simple, flexible structures, easy manufacturing and process and various size and material selection. So the valve requires only simple manufacturing process and flexible construction methods for different patients, a manufacturer for the valve can easily implement rapid product development and production.
(t) To provide an operating-friendly mechanical heart valve, so doctors can operate the valve without specialized tool, the operation will be simpler and easier and less time consuming and less errors. The after-implanting care and monitor will be much easy and controllable.

Still further objects and advantages will become apparent from study of the following description and the accompanying drawings.

### DRAWINGS

Drawing Figures

FIG. 1 is an exploded view of a mechanical heart valve constructed in accordance with this invention.

FIG. 2 is a back view of the mechanical heart valve of FIG. 1 at an open position.

FIG. 3 is a cross view of the mechanical heart valve of FIG. 2 along line A-A.

FIG. 4 is a front view of the mechanical heart valve of FIG. 1 at a closed position

FIG. 5 is a cross view of the mechanical heart valve of FIG. 4 along line B-B.

FIG. 6 is an exploded view of an alternative mechanical heart valve of FIG. 1

FIG. 7 is an exploded view of an alternative mechanical heart valve of FIG. 1

FIG. 8 is a back view of the mechanical heart valve of FIG. 7 at a closed position.

FIG. 9 is a cross view of the mechanical heart valve of FIG. 8 along line C-C.

FIG. 10 is a back view of the mechanical heart valve of FIG. 7 at an open position.

FIG. 11 is a cross view of the alternative body of FIG. 10 along line D-D.

FIG. 12 is an exploded view of an alternative mechanical heart valve of FIG. 1.

FIG. 13 is an exploded view of an alternative mechanical heart valve of FIG. 12.

FIG. 14 is an exploded view of an alternative valve of FIG. 12.

FIG. 15 is a cutoff exploded view of alternative valve of FIG. 1.

FIG. 16 is a cutoff exploded view of an alternative valve of FIG. 1.

FIG. 17 is an exploded view of a mechanical heart valve constructed in accordance with this invention.

FIG. 18 is a front view of the mechanical heart valve with one valve member at an open position and one valve member at a closed position of FIG. 17

FIG. 19 is a cross view of the mechanical heart valve of FIG. 18 along line B-B.

FIG. 20 is a cross view of the mechanical heart valve of FIG. 18 along line A-A.

FIG. 21 is an exploded view of an alternative mechanical heart valve of FIG. 17.

FIG. 22 is an exploded view of an alternative mechanical heart valve of FIG. 17.

FIG. 23 is an exploded view of an alternative mechanical heart valve of FIG. 17.

FIG. 24 is an exploded view of an alternative mechanical heart valve of FIG. 17

FIG. 25 is an exploded view of an alternative mechanical heart valve of FIG. 17

FIG. 26 is an exploded view of an alternative valve of FIG. 17

FIG. 27 is an exploded view of an alternative valve of FIG. 17

FIG. 28 is a cutoff exploded view of an alternative valve of FIG. 17

FIG. 28a is an exploded view of a subassembly of the valve of FIG. 28

FIG. 29 is an exploded view of an alternative mechanical heart valve of FIG. 17

FIG. 30 is a front view of the mechanical heart valve of FIG. 29 with one valve member at an open position and one valve member at a closed position.

FIG. 31 is a cross view of the mechanical heart valve of FIG. 30 along line A-A

FIG. 32 is an exploded view of an alternative mechanical heart valve of FIG. 17

FIG. 33 is a front view of the mechanical heart valve of FIG. 32 with one valve member at an open position and one valve member at a closed position.

FIG. 34 is a cross view of the mechanical heart valve of FIG. 33 along line F-F

FIG. 35 is an exploded view of an alternative mechanical heart valve of FIG. 17

FIG. 36 is an exploded view of a mechanical heart valve constructed in accordance with this invention.

FIG. 37 is a front view of the mechanical heart valve of FIG. 36 with one valve member at an open position and two valve member at a closed position

FIG. 38 is a cross view of the mechanical heart valve of FIG. 37 along line I-I

FIG. 39 is a cross view of the mechanical heart valve of FIG. 37 along line H-H

FIG. 40 is a front view of a leaflet of the mechanical heart valve of FIG. 36

FIG. 41 is a cross view of the leaflet of FIG. 40 along line P-P

FIG. 42 is a front view of a valve body of the mechanical heart valve of FIG. 36

FIG. 43 is a cross view of the valve body of FIG. 42 along line V-V

FIG. 44 is an exploded view of an alternative mechanical heart valve of FIG. 36

FIG. 45 is a front view of the mechanical heart valve of FIG. 44 with one valve member at an open position and two valve member at a closed position.

FIG. 46 is a cross view of the mechanical heart valve of FIG. 45 along line O-O

FIG. 47 is a cross view of the mechanical heart valve of FIG. 45 along line N-N

FIG. 48 is a front view of a leaflet of the mechanical heart valve of FIG. 44

FIG. 49 is a top view of the leaflet of FIG. 48

FIG. 50 is a front view of a valve body of the mechanical heart valve of FIG. 44

FIG. 51 is a cross view of the valve body of FIG. 50 along line W-W

### REFERENCE NUMBER IN DRAWING

| | | | |
|---|---|---|---|
| 100 | valve a, b, c, d, e, f, g, h, k | 151 | leaflet a, b, c, d, e, f, g, h |
| 101 | body a, b, c, d, e, f, g, h | 152 | external surface a, b, c, d |
| 102 | internal surface a, b, c, d | 153 | external surface a, b, c, d |
| 103 | internal surface a, b, c, d | 154 | internal surface a, b, c, d |
| 104 | external surface a, b, c, d | 156 | left side surface a, b, c, d |
| 106 | flow port a, b, c, d | 157 | right side surface a, b, c, d |
| 107 | groove a, b, c, d | 158 | balance section a, b, c, d |
| 108 | front surface a, b, c, d | 159 | actuating section a, b, c, d |
| 109 | back surface a, b, c, d | 160 | leaflet vertical axis a, b, c, d |
| 110 | center axis a, b, c, d | 161 | axis |
| 111 | strip | 165 | first hinge hole a, b, c, d |
| 112 | port g | 166 | second hinge hole a, b, c, d |
| 115 | first hinge hole a, b, c, | 167 | hinge slot a, b, c, d |
| | d, e, f, g | 168 | hinge pin a, b, c, d |
| 116 | second hinge hole a, b, | 169 | hinge pin a, b, c, d |
| | c, d, f, g | 170 | hinge boss a, b, c, d |
| 117 | hinge slot a, b, c, d | 171 | hinge surface a,, b |
| 118 | first hinge pin a, b, c, d, h | 172 | stopper a, b, c, d |
| 119 | second hinge pin a, b, c, | 173 | surface a, b, c, d |
| | d, e, f, g, h | 174 | surface a, b, c, d |
| 120 | hinge boss a, b, c, d | 175 | inward surface a, b, c, d |
| 121 | hinge surface a,, b | 176 | outward surface a, b, c, d |
| 122 | stopper a, b, c, d | 177 | slot a, b, c, d |
| 123 | surface a, b, c, d, | 180 | pivot axis a, b, c, d |
| 124 | surface a, b, c, d | 190 | lock slot a, b, c, d, |
| 125 | inward surface a, b, c, d, e | 191 | lock boss a, b, c, d |
| 126 | outward surface a, b, c, d, e | 192 | internal surface a, b, c, d |
| 127 | slot a, b, c, d | 193 | external surface a, b, c, d |
| 130 | pivot axis a, b, c, d | 194 | surface a, b, c, d |
| 140 | lock slot a, b, c, d | 195 | shaft g |
| 141 | lock boss a, b, c, d | 196 | driver g |
| 142 | internal surface a, b, c, d | 197 | nut f, g |
| 143 | external surface a, b, c, d | 198 | flexible seal h |
| 144 | surface a, b, c, d | 251 | leaflet a, b, c, d, e, f, g, h |
| 145 | spring, e, f, g, h | 252 | external surface |
| 200 | valve a, b, c, d, e, f, g, h | 253 | external surface |
| 201 | body a, b, c, d, e, f, g, h | 254 | internal surface |
| 202 | internal surface a, b, c, d | 256 | left side surface |
| 203 | internal surface a, b, c, d | 257 | right side surface |
| 204 | external surface | 258 | balance section |
| 206 | flow port | 259 | actuating section |
| 207 | groove | 260 | leaflet vertical axial |
| 208 | front surface | 261 | center axis |
| 209 | back surface | 265 | hinge hole a, b, c, d |
| 210 | flow port vertical axis | 266 | hinge hole a, b, c, d |
| 211 | groove | 267 | hinge slot a, b, c, d |
| 215 | hinge hole a, b, c, d, e, | 268 | hinge pin a, b, c, d |
| | f, g, h | 269 | hinge pin a, b, c, d |
| 216 | hinge hole a, b, c, d, h | 270 | hinge boss a, b, c, d |
| 217 | hinge slot a, b, c, d, h | 271 | hinge boss a,, b |
| 218 | hinge pin a, b, c, d, h | 272 | stopper |
| 219 | hinge pin a, b, c, d | 273 | surface |
| 220 | hinge boss a, b, c, d, h | 274 | surface |
| 221 | hinge boss a, b, c, d, h | 275 | inward surface |
| 222 | stopper | 276 | outward surface |
| 223 | surface | 277 | slot a, b, c, d, e |
| 224 | surface | 280 | pivot axis |
| 225 | inward surface | 290 | lock slot |
| 226 | outward surface | 291 | lock boss |
| 227 | slot a, b, c, d, e | 292 | internal surface a, b |
| 230 | pivot axis | 293 | external surface a, b |
| 240 | lock slot | 294 | cylindered surface |
| 241 | lock boss | 246 | set screw, h |
| 242 | internal surface a, b | 296 | L block |
| 243 | external surface a, b | 351 | leaflet a, b |
| 244 | cylindered surface | 352 | external surface |
| 245 | spring | 353 | external surface |
| 300 | valve a, b | 354 | internal surface |
| 301 | body a, b | 356 | left side surface |
| 302 | internal surface | 357 | right side surface |
| 303 | internal surface | 358 | balance section |
| 304 | external surface | 359 | actuating section |
| 306 | flow port | 360 | leaflet vertical axis |
| 307 | groove | 361 | axis |
| 308 | front surface | 365 | hinge hole a, b, c, d |
| 309 | back surface | 366 | hinge hole a, b, c, d |
| 310 | flow port axis | 367 | hinge slot a, b, c, d |
| 315 | hinge hole a, b, c, d | 368 | hinge pin a, b, c, d |
| 316 | hinge hole a, b, c, d | 369 | hinge pin a, b, c, d |
| 317 | hinge slot a, b, c, d | 370 | hinge boss a, b, c, d |
| 318 | hinge pin a, b, c, d | 371 | hinge boss a,, b |
| 319 | hinge pin a, b, c, d | 372 | stopper |
| 320 | hinge boss a, b, c, d | 373 | surface |
| 321 | hinge boss a, b, c, d | 374 | surface |
| 322 | stopper | 375 | inward surface |
| 323 | surface | 376 | outward surface |
| 324 | surface | 377 | slot a, b, c, d |
| 325 | inward surface | 380 | pivot axis |
| 326 | outward surface | 390 | lock slot |
| 327 | slot a, b, c | 391 | lock boss |
| 330 | pivot axis | 392 | internal surface a, b |
| 340 | lock slot | 393 | external surface a, b |
| 341 | lock boss | 394 | surface |
| 342 | internal surface a, b | 451 | leaflet a |
| 343 | external surface a, b | 452 | external surface a |
| 344 | surface | 453 | external surface a |
| 345 | Spring | 454 | internal surface a |
| 400 | valve a | 456 | left side surface a |
| 401 | body a | 457 | right side surface a |
| 402 | internal surface a | 458 | balance section a |
| 403 | internal surface a | 459 | actuating section a |
| 404 | external surface a | 460 | leaflet vertical axis a |
| 406 | flow port a | 461 | center axis a |
| 407 | groove a | 465 | first hinge hole a, |
| 408 | front surface a | 466 | second hinge hole a |
| 409 | back surface a | 467 | hinge slot a |
| 410 | flow port vertical axis a | 468 | first hinge pin a, |
| 411 | block a | 469 | second hinge pin a, |
| 415 | first hinge hole a | 470 | first hinge boss a, |
| 416 | second hinge hole a, | 471 | second hinge boss a, |
| 417 | hinge slot a, | 472 | stopper |
| 418 | first hinge pin a | 473 | stopper left surface |
| 419 | second hinge pin a, | 474 | stopper right surface |
| 420 | first hinge boss a, | 475 | inward surface |
| 421 | second hinge boss a | 476 | outward surface |
| 422 | stopper | 477 | hinge slot a |
| 423 | stopper left surface | 480 | pivot axis |
| 424 | stopper right surface | 481 | hinge slot a, |
| 425 | inward surface | 490 | lock slot |
| 426 | outward surface | 491 | lock boss |
| 427 | hinge slot a, | 492 | internal surface a, b |
| 430 | pivot axis a | 493 | external surface a, b |
| 431 | hinge slot a, | 494 | surface |
| 440 | lock slot | 495 | release slot a |
| 441 | lock boss | | |
| 442 | internal surface a, | | |
| 443 | external surface a, | | |
| 444 | surface | | |

### DESCRIPTION

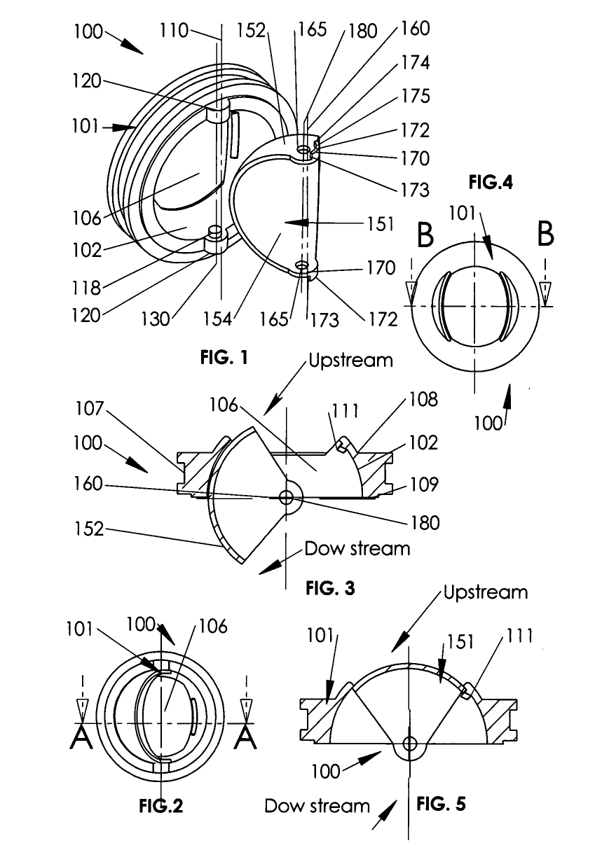

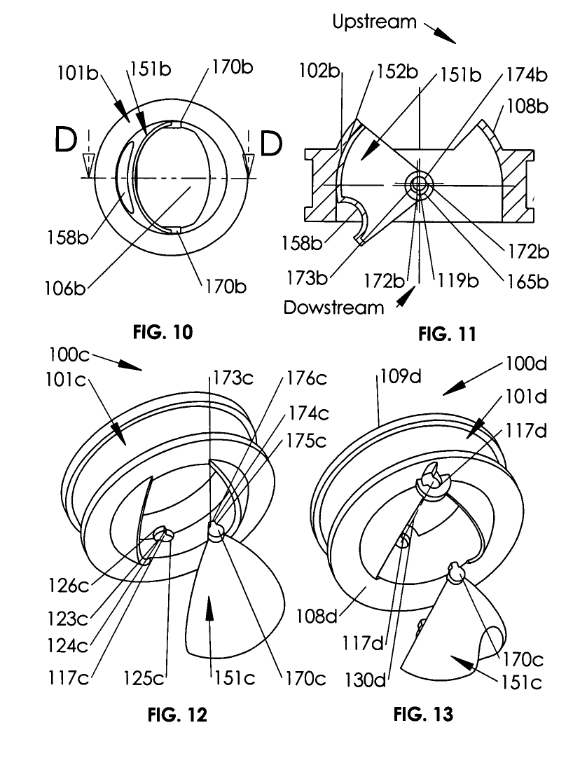

FIGS. 1-16 illustrate a mechanical heart valve 100 and other alternative embodiments in accordance with the present invention. Valve 100 disposed in a native heart (not shown) by means of a groove 107 comprises a body 101 having a flow port 106 between a front surface 108 and a back surface 109 and a valve member or leaflet 151. The leaflet 151 movably disposed in the flow port 106 is actuated by pressure difference between upstream flow and downstream flow to regulate blood flow through port 106 between closing and opening positions.

Referring now to FIGS. 1-5. The body 101 has a spherical internal surface 102 with a center axis 110 and two hinge bosses 120 defined by an eccentric pivot axis 130 on port 106 in an opposite direction. Each of hinge bosses 120 has a concentric hinge pin 118. The leaflet 151 is movably disposed in port 106 by means of a spherical shell surface 152 defined by a centric axis 160 and two hinge bosses 170 defined by an eccentric pivot axis 180. Each boss 170 has a hinge hole 165 engaged respectively with hinge pin 118. A profile diameter of surface 102 is substantially the same as that of surface 152, the pivot axis 180 is constantly concentric with pivot axis 130. When leaflet 151 is in an open position, axis 160 is away from center axis 110 in body 101, so the valve 100 has a partial engagement and seal between surface 102 and surface 152, whereas when leaflet 151 is at a closed position, axis 160 is concentric with axis 110 in body 101, so the valve 100 has a full engagement and seal between surfaces 102, 152. Valve body 101 also provides a strip 111 to prevent an overtravel of leaflet 151 and cavitations. Each boss 170 also comprises a cylindrical stoppers 172 in an opposite direction around hinge holes 165, each stopper 172 is defined by two surfaces 173, 174 to prevent overtravel of leaflet 151, surface 173 is against surface 109 when the leaflet 151 is at a closed position, while surface 174 is against surface 109 when the leaflet 151 is at an open position.

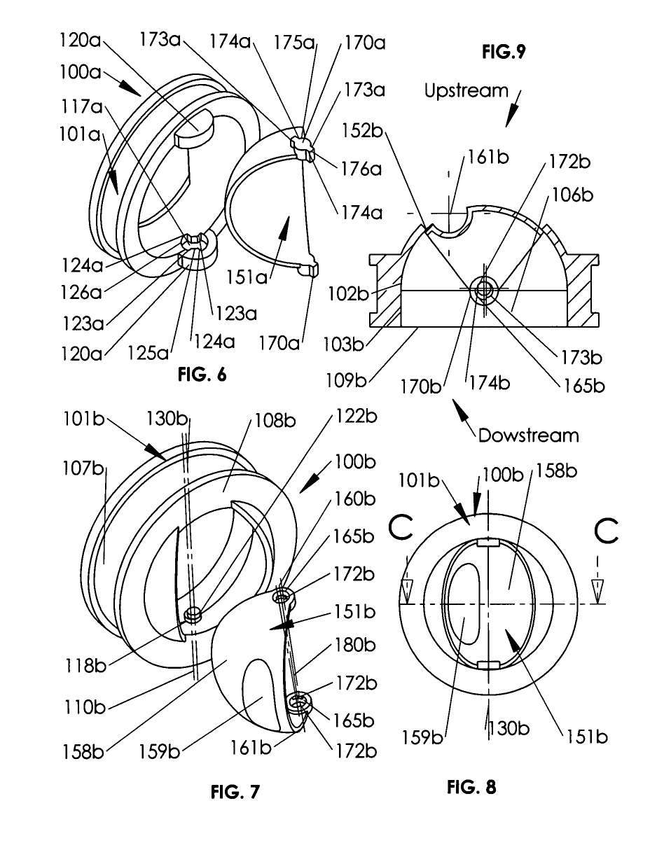

Referring now to FIG. 6, a valve 100a is an alternative of valve 100. The valve 100a has a body 101a and a leaflet 151a with an alternative hinge mechanism. The body 101a has two hinge bosses 120a in an opposite direction. Each of hinge bosses 120a has a concentric hinge slot 117a. The hinge slot 117a is defined by two partial cylindrical surfaces 125a, 126a and two surfaces 123a, 124a. Two hinge bosses 170a in leaflet 151a are movably, respectively engaged with hinge slots 117a, each hinge boss 170a is defined by two cylindrical surfaces 175a, 176a and two stopping k765surfaces 173a,174a. When leaflet 151a is at a closed position, surface 174a is against overtravel of surface 124a, while when leaflet 151a is at an open position, surface 173a is against overtravel of surface 123a.

Referring now to FIGS. 7-11, a valve 100b is an alternative of valve 100. The valve 100b has a body 101b and a leaflet 151b. The body 101b has a spherical internal surface 102b with a centric axis 110b and a cylindrical surface 103b and two hinge pins 118b defined by axis 110b on port 106b in an opposite direction. Each of hinge pin 118b has an eccentric, cylindrical stopper 122b defined by an axis 130b. The leaflet 151b comprises a spherical balanced section 158b having a centric axis 160b and a cylindrical actuation section 159b having a centric axis 161b and is movably disposed in body 101b by means of a spherical shell surface 152b engaged with surface 102b and two hinge holes 165b engaged respectively with hinge pin 118b. A profile diameter of ports 102b is substantially the same as that of surface 152b, each hinge hole 165b has two eccentric, partial cylindrical stopper 173b defined by two axils 180b in an opposite direction, a diameter of stopper 172b is substantially the same as that of stopper 122b. A distance between axis 110b and 130b is substantially the same as that of between axils 160b and 180b, axis 110b in body 101b is constantly concentric with axis 160b. When valve 100b is at an open position, stopper 122b is concentric with axis 180b of one of stoppers 172b, while valve 100b is at a closed position, stopper 122b is concentric with axis 180b of one of stoppers 172b, so overtravel of leaflet 151b is prevented at both closed and open positions.

Referring now to FIG. 12, a valve 100c is an alternative of valve 100 with an alternative hinge mechanism. The valve 100c has a body 101c and a leaflet 151c, body 101c has two hinge slots 117c in an opposite direction. Each of hinge slots 117c comprises a partial inward cylindrical surface 125c and an outward cylindrical surface 126c connected by stopping surfaces 123c,124c. The leaflet 151c is movably disposed in body 101c by means of two hinge bosses 170c engaged respectively with hinge slots 117c, each hinge boss 170c has two cylindrical surfaces 175c,176c connected by two stopping surfaces 173c,174c. When leaflet 151c is at a closed position, surface 173c is against surface 123c to prevent overtravel of leaflet 151c, on the other hand when leaflet 151c is at an open position, surface 174c is against surface 124c to prevent overtravel of leaflet 151c.

Referring now to FIG. 13, a valve 100d is an alternative of valve 100c with an alternative hinge mechanism, valve 100d has a body 101d and leaflet 151c, body 101d has two hinge slots 117d respectively located on a front surface 108d and a back surface 109d defined by a title pivot axis 130d, leaflet 151c is movably disposed in body 101d by means of hinge bosses 170c.

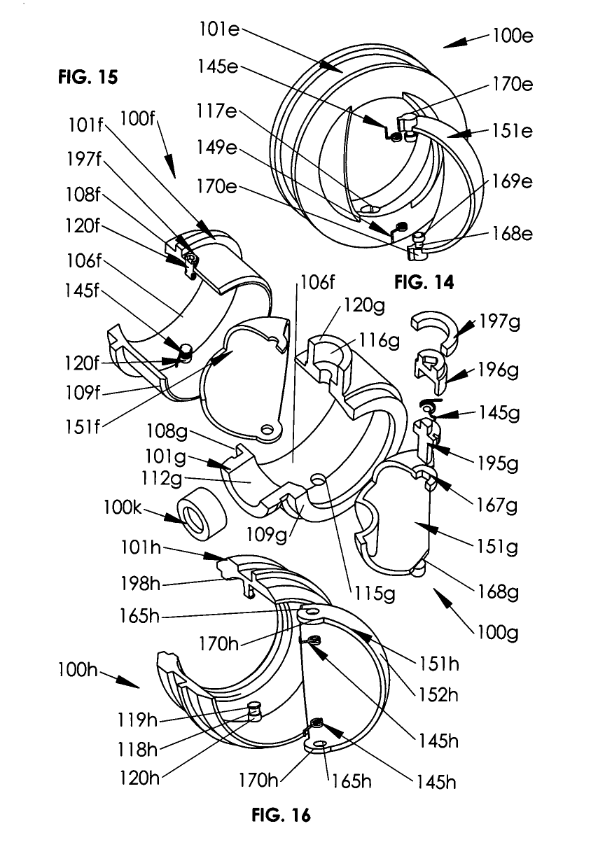

Referring now to FIG. 14, a valve 100e is an alternative of valve 100c with an alternative hinge mechanism, valve 100e has a body 101e and a leaflet 151e. body 101e has two hinge slots 117e in an opposite direction. The leaflet 151e has two hinge boss 170e engaged respectively with hinge pin 117e, each hinge boss 170e has two hinge pins 168e,169e. Two spring 145e are respectively disposed around pin 168e, pin 168e is smaller than pin 169e to prevent spring 145e to fall out. A closing torque is provided with one end of spring 145e biased against slot 117e and other end of spring 145e biased against a slot of pin 169e.

Referring now to FIG. 15, valves 100f, 100g are alternatives of valve 100 with two bodies 101f,101g and two leaflets 151f, 151g, a valve 100k is a conventional linear pressure relief valve. A combination of those valve in series, parallel or mix can provide multiple functions valves. A valve 100fg has valve 100f as a check valve and valve 100g as a pressure regulator for a drainage system, a valve 100gk has valve 100g as a check valve and valve 100k as a relief valve for a drainage system, valve 100g can be used as a two-way check valve.

The valve 100f is based on the offset pressure porting mechanism and has body 101f and leaflet 151f movably disposed in body 101f for porting flow through a port 106f, body 101f has two hinge bosses 120f with thread joints, each boss 120f is provided with a springs 145f and a nut 197f for a closing torque. Nut 197f is provided for adjusting the closing torque and securing the boss 120f. Body 101f also has two thread joints on a surface 109f to connected with body 101g or other device and on a surface 108f to connected with a drainage container with a filter or a inlet tube (not shown).

The valve 101g is based on the hydride function pressure porting mechanism and has body 101g and leaflet 151g movably disposed in body 101g for porting a flow through a port 106g, a surface 108g on port 10g has a thread joint for connecting with body 101f or others. Body 101g has a port 112g, a lower hinge hole 115g extending to a upper hinge through hole 116g and a large hinge boss 120g, leaflet 151g is movably disposed in body 101g by means of a hinge pin 168g disposed in hole 115g and hinge hole 167g to received a shaft 195g , one end of shaft 195g is disposed in hinge hole 115g to engaged with slot 167g, other end of shaft 195g is movably disposed in hole 116g . Valve 101g also has a spring 145g and a driver 196g, an operating torque is provided with one end of spring 145g biased against a slot of shaft 195g and other end of spring 145g biased against a driver 196g threaded with hole 116g. A nut 197g threaded with driver 196g is to secure a position of driver 196g. The driver 196g can be operated by manual, motor or others to control a presetting torque.

A valve 100fg is a combination of valves 100f, 100g with the thread joint between surfaces 108g and 109f to provide a drainage system. A valve 100gk is a combination of valves 100g, 100k with the thread joint in hole 112g to provide a drainage system in heart operation. Valve 100g can be used as a two-way check valve with two inlets ports defined by hole 112g and port 106g on surface 109g and one outlet port defined by port 106g on surface 108g.

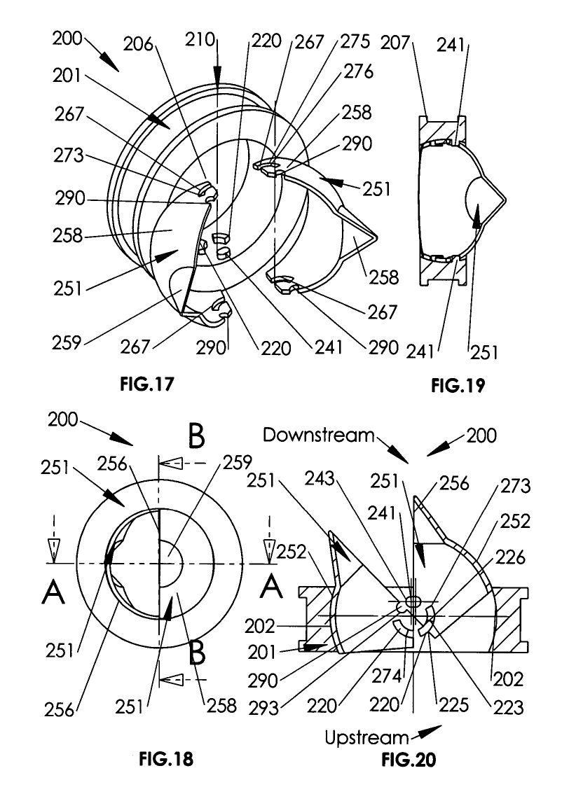

Referring now to FIG. 16, a valve 100h is an alternative of valve 100c with a body 101h and a leaflet 151h is used as a check valve or pressure regulator for a fuel delivery system. The body 101h is constructed to be inserted into a pipe or tube of the fuel delivery system, body 101h has also a bonded flexible seal 198h and two hinge boss 120h in an opposite direction. Each boss 120h has two hinge pins 118h,119h, pin 118h is smaller than pin 119h to prevent any spring ground pin 118h to fall out. The leaflet 151h has two hinge boss 170h comprising respectively hinge slot 165h in an opposite direction and is movably disposed in body 101h by means of engagement between seal 198h and a spherical shell surface 152h, between hinge bosses 120h and hinge slots 165h. Two springs 145h are respectively disposed around pins 118h, a presetting torque is provided with one end of spring 145h biased against hinge boss 170h and other end of spring 145h biased against a slot of pin 119h to control a presetting pressure of the fuel delivery system.

FIGS. 17-28a illustrate a mechanical heart valve 200 and other alternative embodiments in accordance with the present invention. Valve 200 disposed in a native heart (not shown) by means of a groove 207 comprises a body 201 having a flow port 206 between a front surface 208 and a back surface 209 and two valve members or leaflets 251. The leaflets 251 disposed in the flow port 206 is actuated by pressure difference between upstream flow and downstream flow to regulate blood flow through port 206 between closed and open positions.

Referring now to FIGS. 17-20, valve 200 has a body 201 and two leaflets 251. The body 201 has a spherical internal surface 202 with a centric axis 210 and four pivot hinge bosses 220 defined by axis 210 on port 206 and two lock bosses 241 in an opposite direction. Each of hinge bosses 220 is defined by two cylindrical surfaces, 225, 226 and two flat surfaces 223, 224. Each lock boss 241 has two eccentric cylindrical surfaces 243. The leaflet 251 comprises a spherical balanced section 258 having a centric axis 260 and a conical actuation section 259 and is movably disposed in port 206 by means of a spherical shell surface 252 engaged with surface 202 and two hinge slot 267 engaged respectively with hinge bosses 220. A profile diameter of surface 202 is substantially the same as that of surface 252, each hinge slot 267 is defined by two cylindrical surfaces 275, 276 and surface 273, leaflet 251 also a partial cylindrical lock slot 290, a diameter of slot 290 is slightly larger than that of surface 243, but an opening of lock slot 290 is smaller than that of surface 243, so when valve leaflet 251 is at an open position, surface 273 is against surface 223 to prevent any overtravel of leaflet 251, when leaflet 251 is approached to a closed position, first lock slot 290 impacts the lock boss 242, because the opening of slot 290 is smaller than profile diameter of surface 243, a snap action takes place, lock boss 241 gets in lock slot 290, then two side surfaces 256 of leaflets 251 contact each other to provide a seal.

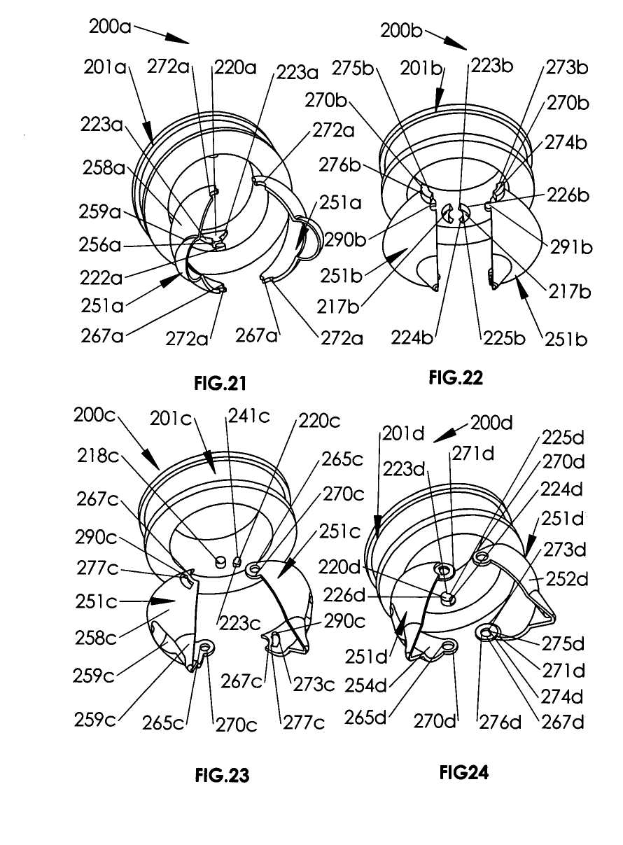

Referring now to FIG.21, a valve 200a is an alternative of valve 200. Valve 200a has a body 201a and two leaflets 251a. The body 201a has two hinge boss 220a and two stoppers 222a in an opposite direction. Each hinge boss 220a is defined by two surfaces 223a and a cylindrical surface 256a, leaflet 251a has a spherical balanced section 258a and a spherical actuation section 259a and is movably disposed in body 201a by means of two hinge slots 267a respectively engaged with hinge boss 220a, leaflet 251a also has two stop slots 272a . When leaflet 251a is at a closed position, the stopper 222a is provided to stop leaflet 251a to overtravel, when leaflet 251a is at open position, the surface 223a is provided to stop leaflet 251a to overtravel.

Referring now to FIG. 22, a valve 200b is an alternative of valve 200 with an alternative hinge mechanism. The valve 200b has a body 201b and two leaflets 251b, body 201b has four hinge slots 217b, two of which are in an opposite direction. Each hinge slot 217b is defined by two cylindrical surfaces 225b , 226b and two stopping surfaces 223b, 224b. The leaflet 251b movably disposed in body 201b has two hinge bosses 270b and a lock boss 291b and a lock slot 290b in an opposite direction. The hinge boss 270b movably engaged with 217b is defined by two cylindrical surface 275b, 276b and two flat surfaces 273b and 274b. A diameter of lock slot 290b is slightly larger than that of boss 291b, but an opening of lock slot 290b is slightly smaller than that of lock boss 291b, so when leaflet 251b is approached to a closed position, the lock boss 291b first impacts lock slot 290b, a snap action takes place, then side surfaces of leaflets 251b contact either other and provide a seal, surfaces 224b, 223b are provided to prevent over-travel of leaflet 251b.

Referring now to FIG. 23, a valve 200c is an alternative of valve 200. Valve 200c has a body 201c and two leaflets 251c, body 201c has two cylindrical hinge pin 218c and two cylindrical hinge boss 220c in an opposite direction. Each hinge boss 220c is defined by a lock boss 241c and a surfaces 223c. The leaflet 251c comprises a spherical balanced section 258c and an actuation section 259c defined by conical, cylindrical and spherical profiles. The leaflet 251c movably disposed in body 201c has a cylindrical hinge boss 270c with a concentric holes 265c on one side and two cylindrical hinge slots 267c, 277c in an opposite side. Each hinge holes 265c is movably engaged with hinge pin 218c, each hinge slot 267c movably engaged with hinge boss 270c, hinge slot 277c engaged with hinge boss 220c has also a lock slot 290c and stopping surface 273c. A diameter of lock slot 290c is slightly larger than that of boss 241c, but an opening of lock slot 290c is slightly smaller than that of lock boss 241c, so when leaflet 251c is approached to a closed position, the lock slot 290c first impacts lock boss 241c, a snap action takes place, then side surfaces of leaflets 251c contact either other and seal, when leaflet 251c is at an open position, the surface 273c is against surface 223c to prevent leaflet 251c to overtravel.

Referring now to FIG. 24, a valve 200d is an alternative of valve 200, valve 200d has a body 201d and two leaflets 251d. The body 201d has two cylindrical hinge boss 220d in an opposite direction. Each hinge boss 220d is defined by two cylindrical surfaces 226d, 225d and two stopping surfaces 223d, 224d, The leaflet 251d comprises a spherical balanced section 258d and an actuation section 259d defined by a conical, a cylindrical and a spherical profiles. The leaflet 251d movably disposed in body 201d has a cylindrical hinge boss 270d along with an external surface 252d and a cylindrical hinge boss 271d along with an internal surface 254d in an opposite direction, the hinge boss 270d has a hinge pin hole 265d , while hinge boss 271d has a hinge slot 267d defined by two cylindrical surface 275d, 276d and two stopping surfaces 273d,274d. Two hinge bosses 270d, 271d are movably overlapped and engaged with hinge pin 220d, so when leaflet 251d is at a closed position, side surfaces of leaflets 251c contact either other, then surface 223d is against 273d to stop leaflet 251d, when leaflet 251d is at an open position, the surface 224d is against surface 274d to stop leaflet 251d.

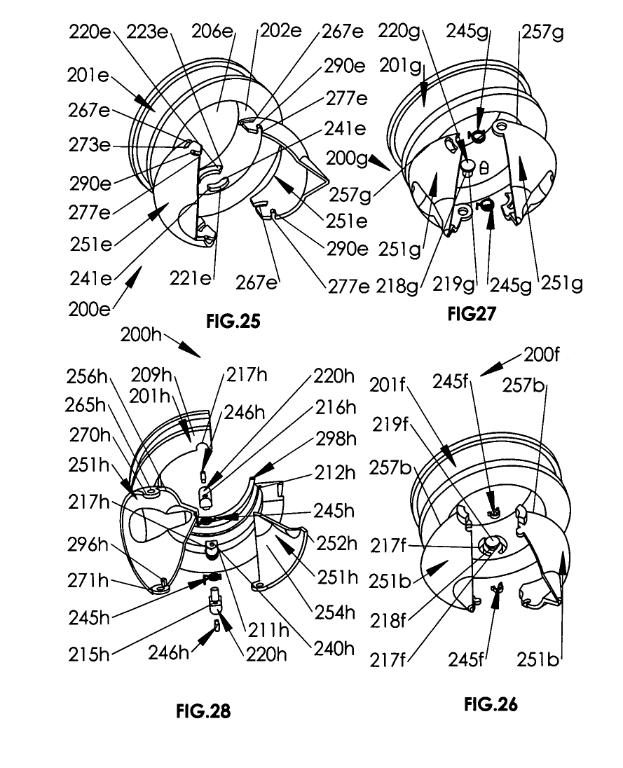

Referring now to FIG. 25, a valve 200e is an alternative of valve 200. The valve 200e has a body 200e and two leaflets 251e. The body 201e has a spherical internal surface 202e and four cylindrical hinge bosses 220e, 221e on a port 206e in an opposite direction. Each hinge boss 220e is defined by two surfaces 223e, each hinge boss 221e also has two lock bosses 241e. The leaflet 251e is movably disposed in a port 206e by means of four hinge slots 267e, 277e engaged respectively with hinge bosses 220e, 221e. Each hinge slot 267e has a stopping surface 273e, while each hinge slot 277e also has a lock slot 290e. A diameter of lock slot 290e is slightly larger than that of lock boss 241e, but an opening of 290e is slightly smaller than that of lock boss 241e. When leaflet 251e is approached to a closed position, first lock slot 290e impacts the lock boss 241e, a snap action takes place, lock boss 241e gets in lock slot 290e, then side surfaces of leaflets 251e contact each other and provide a seal, when leaflet 251e is at an open position, surface 223e is provide to prevent any overtravel of leaflet 251e.

Referring now to FIG. 26, a valve 200f is an alternative of valve 200b with an alternative hinge mechanism. The valve 200f has a body 201f and two leaflets 251b. Body 201f has four hinge slot 217f, two of which are in an opposite direction body 201f has also two cylindrical hinge pins 218f in an opposite direction, each hinge pin 218f has a centric hinge pin 219f, a diameter of pin 218f is smaller than that of 219f to prevent any spring to fall out. Two omega springs 245e are respectively disposed around pins 218e , a closed torque is provided with both ends of spring 245f biased against side surfaces 257b.

Referring now to FIG. 27, a valve 200g is an alternative of valve 200c, with an alternative hinge mechanism. The valve 200g has a body 201 g and two leaflets 251g, two hinge bosses 220g are connected body 201g with threat joints in an opposite direction, each hinge boss 220g has a pin 218g and a larger pin 219g, two torsion springs 245g are respectively disposed around pins 218g . A closed torque is provided with both ends of spring 245g biased against side surfaces 257g.

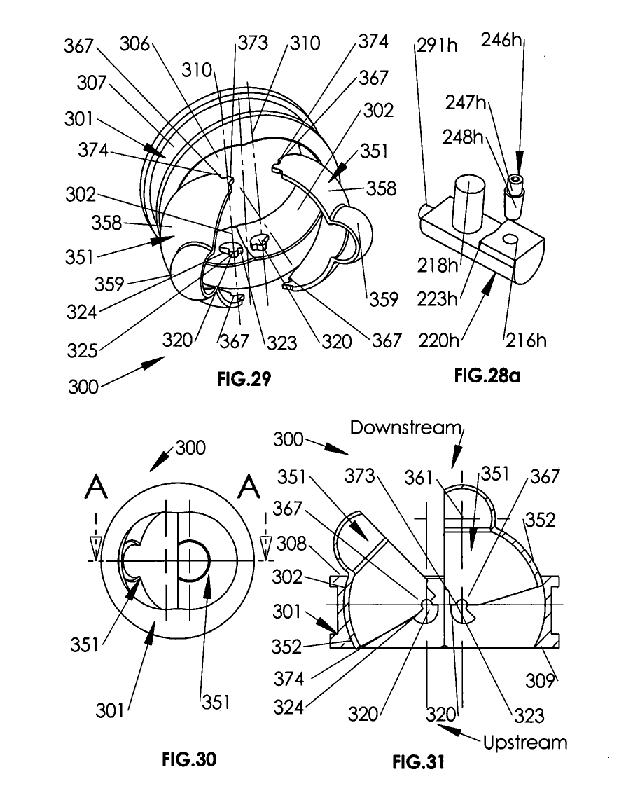

Referring now to FIGS. 28-28a, a valve 200h is an alternative of valve 200d and used as a dual disc check valve in industrial as well as commercial markets. Valve 200h has a body 201h and two leaflets 252h, body 210h has two cylindrical hinge slots 217h on a surface 209h in an opposite direction, each slot 217h has a concentric, conical groove 211h and a lock cylindrical slot 240h, an opening of slot 217h is smaller than a diameter of the slot 217h. Each hinge boss 220h has a stopping surface 223h and a cylindrical lock boss 291h inserted into the slot 240h, hinge boss 220h has also an eccentric hinge pin 218h and a larger holes 216h extended to an outward smaller thread hole 215h engaged with an end 247h of a setscrew 246h, other conical end 246h of screw 246h is engaged with groove 211h to secure boss 220h. The leaflet 251h movably disposed in body 201h has an eccentric cylindrical hinge boss 270h along with an external surface 252h and an eccentric cylindrical hinge boss 271h along with an internal surface 254h in an opposite direction. Hinge bosses 270h and 271h respectively have two hinge hole 265h engaged with hinge pin 218h in an overlapping manner. Body 201h also has a tapped groove to secure a O-ring 246h to provide a seal between body 201h and leaflets 251h. Side surface 256h of leaflet 251h are coated with rubber or plastics to provide a seal between leaflets 251h. When leaflet 251h is in an open position, a center axis of 251h is away from a center axis of body 201h, so the valve 200h has a partial engagement and seal between body 201h and leaflet 251h, whereas when leaflet 251h is at a closed position, the center axis of leaflet 251h is concentric with the center axis of body 201h, so the valve 200h has a full engagement and seal between body 202h and leaflet 251h. Valve 200h also has two torsion springs 245h, each spring 245h is disposed between pin 218h and a L-block 296h, a closing torque is provided with both ends of spring 245h biased against two leaflets 251h.

FIGS. 29-35 illustrate a mechanical heart valve 300 and other alternative embodiments in accordance with the present invention. Valve 300 disposed in a native heart (not shown) by mean of a groove 307 comprises a body 301 having a flow port 306 between a front surface 308 and a back surface 309 and two valve members or leaflets 351. The leaflets 351 disposed in the flow port 306 is actuated by pressure difference between upstream flow and downstream flow to regulate blood flow through port 306 between closing and opening positions.

Referring now to FIGS. 29-31, valve 300 has a body 300 and two leaflets 351. The body 301 has two spherical internal surfaces 302 equally off from a center of flow port 306 with two axils 310 and four hinge bosses 320, two of hinge bosses 320 located on a opposite direction. Each hinge boss 320 is defined by a cylindrical surface 325 and two flat surfaces 323, 324. The leaflet 351 comprises a spherical balanced section 358 and a spherical actuation section 359 having a centric axis 361 and is movably disposed in port 306 by means of a spherical shell surface 352 engaged with surface 302 and two hinge slot 367 engaged respectively with hinge bosses 320. A profile diameter of surface 302 is substantially the same as that of surface 352, so when leaflet 351 is at an open position, surface 374 is against surface 324 to prevent an overtravel of leaflet 351, when leaflet 351 is at a closed position, surface 373 is against surface 323 to prevent an overtravel of leaflet 351.

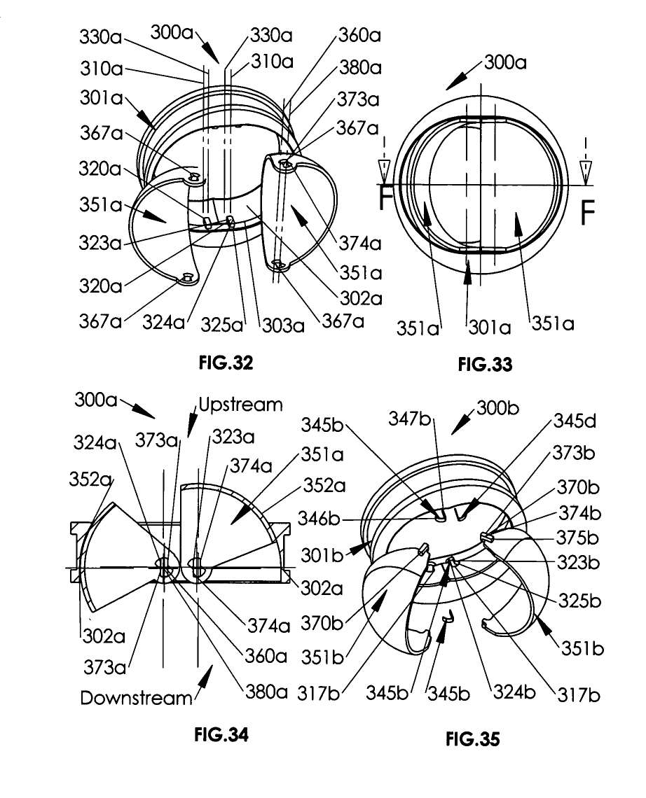

Referring now to FIGS. 32-34, a valve 300a is an alternative of valve 300. The valve 300a has a valve body 301a and two leaflet 351a. The body 301a has two spherical internal surfaces 302a equally off from a center of flow port extended to two cylindrical surfaces 303a with centric axils 310 and four eccentric hinge bosses 320a defined by two pivot axils 330a. Each of hinge bosses 320a is defined by a cylindrical surface 325a and two flat surfaces 323a, 324a. Each leaflet 351a is movably disposed in body 301a by means of a spherical shell surface 352a engaged with surface 302a and two eccentric hinge slots 367a engaged respectively with hinge boss 320a, a pivot axis 380a between two slots 367a is concentric with pivot axis 330a. A profile diameter of surface 302a is substantially the same as that of surface 352a with an center axis 360a. When leaflet 351a is at an open position, axis 360a is away from center axis 310a in body 301a, surfaces 323a, 324a are against surface 373a to prevent an overtravel of leaflet 351a, so the valve 300a has a partial engagement and seal between surface 302a and surface 352a, whereas when leaflet 351a is at a closed position, axis 360a is concentric with axis 310a in body 301a, surfaces 323a, 324a are against surface 374a to prevent an overtravel of leaflet 351a, so the valve 300a has a full engagement and seal between surface 302a and surface 352a.

Referring now to FIG. 35, a valve 300b is an alternative of valve 300a with an alternative hinge mechanism. The valve 300b has a body 301b and two leaflets 351b, body 301b has four hinge slots 317b in an opposite direction. Each hinge slot 317b is defined by two cylindrical surfaces 325b and two flat surfaces 323b, 224b. The leaflet 351b movably disposed in body 301b has two hinge bosses 370b in an opposite direction, the hinge boss 370b movably engaged with slot 317b is defined by two cylindrical surfaces 375b and two flat surfaces 372b and 373b, four L-shape springs 345b are respectively disposed in hinge slots 317b, a closing torque is provided by one end of spring 345b biased against surface 324b and other end of spring 345b biased against flat surface 323b. When leaflet 351b is approached to open position, the hinge boss 370b is moving against end 347b of spring 345b until spring 345b to contact surface 323b. When leaflet 351b is approached to a closed position, leaflet 351b is actuated by both flow and bended spring 345b.

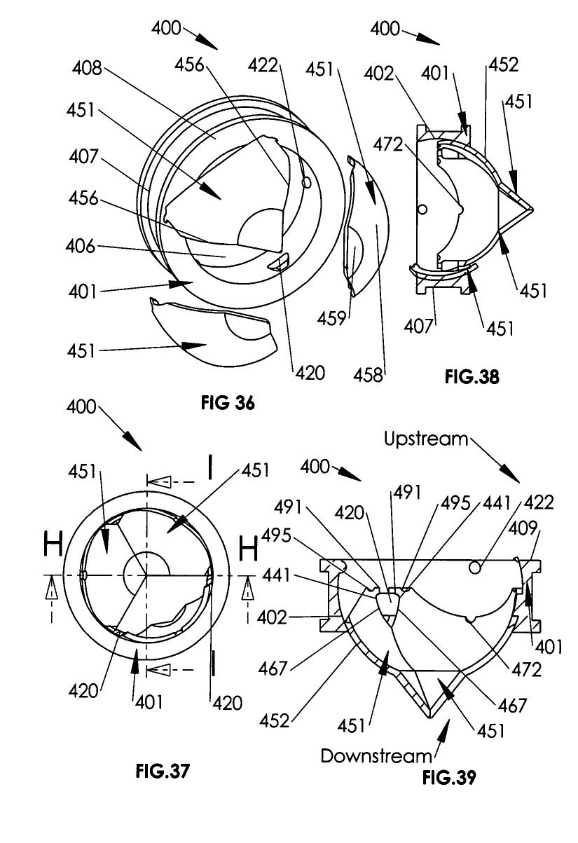

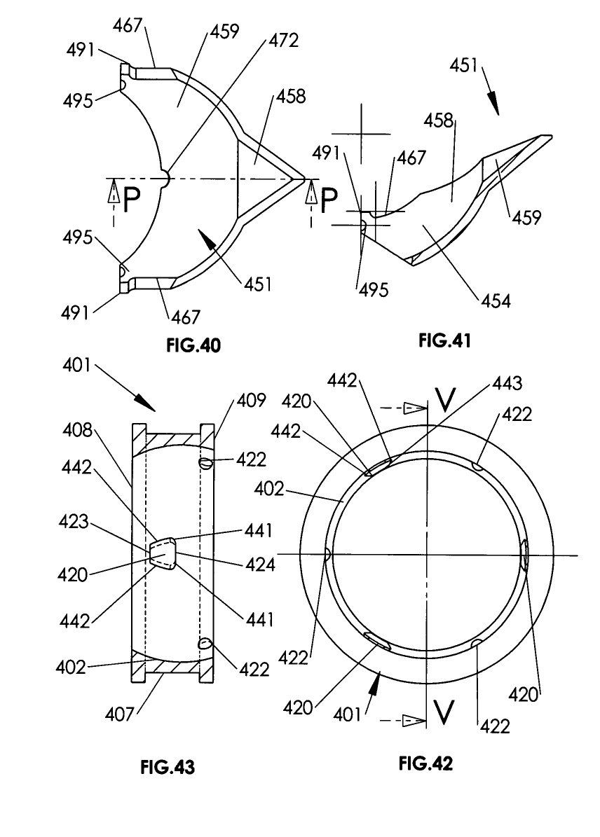

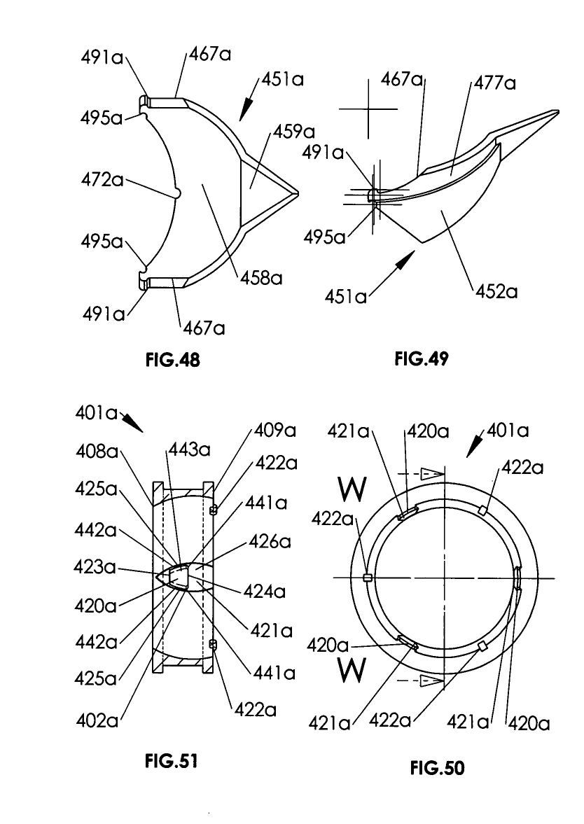

FIGS. 36-51 illustrate a mechanical heart valve 400 and other alternative embodiment in accordance with the present invention. Valve 400 disposed in a native heart (not shown) by mean of a groove 407 comprises a body 401 having a flow port 406 between a front surface 408 and a back surface 409 and three valve members or leaflets 451. The leaflets 451 disposed in the flow port 406 is actuated by pressure difference between upstream flow and downstream flow to regulate blood flow through port 406 between closing and opening positions.

Referring now to FIGS. 36-43, valve 400 has body 401 and three leaflets 451. The body 401 has a spherical internal surface 402 and three hinge bosses 420 and three spherical stoppers 422 equally spanned on surface 402, Each hinge boss 420 is defined by a top spherical internal surface 443 and two side cylindrical surfaces 442 and two flat surfaces 423, 424 and two lock corners 441. The leaflet 451 comprises a spherical balanced section 458 and a conical actuation section 459 and is movably disposed in port 406 by means of a spherical shell surface 452 engaged with surface 402 and two hinge slots 467 engaged respectively with hinge bosses 420. A profile diameter of surface 402 is substantially the same as that of surface 452, a profile diameter of hinge slot 467 is substantially the same as that of surface 442 of hinge boss 422. The leaflet 451 also has two lock bosses 491 and two release slots 495 in an opposite direction and a stopper slot 472, so when valve leaflet 451 is at an open position, stopper slot 472 is against stopper 422 to prevent any overtravel of leaflet 451, when leaflet 451 is approached to a closed position, first lock boss 491 impacts the lock corners 441, because there is release slot 477, lock boss 490 is further compressed to a side and locked with corner 441, then side surfaces 456 of leaflets 451 contact each other and provide a seal.

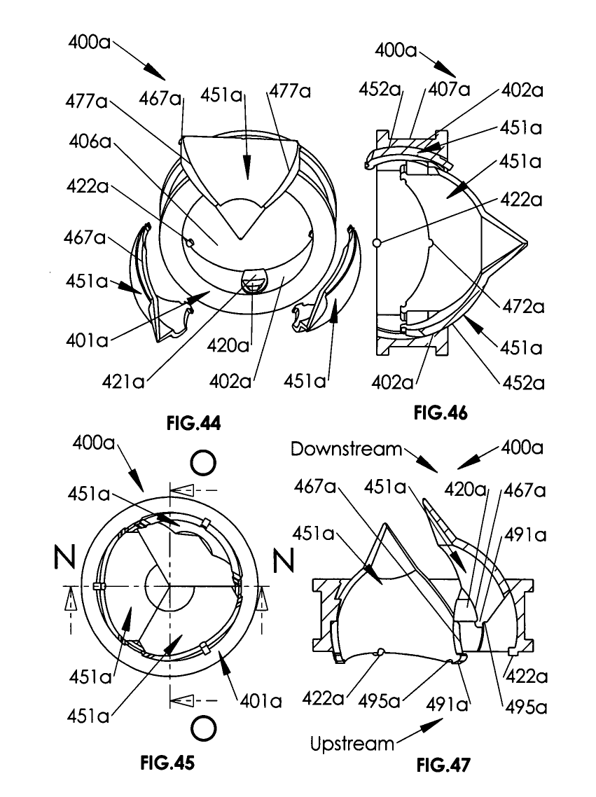

Referring now to FIGS. 44-51, a valve 400a is an alternative of valve 400 with an alternative hinge mechanism. The valve 400a has a body 401a and three leaflets 451a . The body 401 a has a spherical internal surface 402a and three hinge bosses 421a and three cylindrical stoppers 422a equally spanned on surface 402a, Each hinge boss 421a is defined by a top spherical surface 426a and two cylindrical surfaces 225a, hinge boss 421a also has a concentric hinge boss 420a defined by a top spherical internal surface 443a and two side cylindrical surfaces 442a and two flat surfaces 423a, 424a and lock corners 441a. The leaflet 451a comprises a spherical balanced section 458a and a conical actuation section 459a and is movably disposed in a port 406a by means of a spherical shell surface 452a engaged with surface 402a, two hinge slots 467a engaged respectively with hinge bosses 420a and two hinge slots 477a engaged respectively with two hinge bosses 421a. A profile diameter of surface 402a is substantially the same as that of surface 452a, a profile diameter of hinge slot 467a is substantially the same as that of surface 442a of boss 420a, a profile diameter of hinge slot 477a is substantially the same as that of surface 425a of boss 421a. The leaflet 451a also has two lock boss 491a and two release slots 495a in an opposite direction, so when leaflet 451a is at an open position, slot 472a is against stopper 422a to prevent any overtravel of leaflet 451a, when leaflet 451a is approached to a closed position, first lock boss 491a impacts the lock corners 441a, because there is release slot 495a, lock boss 491a is compressed to a side and locked with corner 441a, then side surfaces 453a contact each and provide a seal.

Valve 100,200,300,400 and their alternatives can be constructed with a plurality of materials and a plurality of process for those materials The materials include metals, plastic, rubber, composite, carbon, metal coated with rubbing, plastic, carbon, ceramic and others. While the processes of those materials comprised casting, forging, molding, fabrication, stamping, bonding, machining, welding, assembly, sterilizing and others. The surfaces for contact surfaces in medical valve must be coated or treated with antifriction, rubbing material or anticoagulant drug, above all those materials for medical usage must be bio-compatible and safety. So the processes and materials should be at low cost and easy to use and flexible to be substitute.

Valve 100,200,300,400 and their alternatives are constructed with a plurality of configurations, the configurations of connecting ends include a tube adapter end, flange, wafer, lug, spited body end, welded end, inline end, threaded end, grooved end, angle body end. The configurations of the flow port comprises at least one inlet and one outlet. The configurations of functions include; check valve/check valve, check valve/pressure relief valve, check valve/pressure regulator, relief valve/relief valve, relief valve/pressure regulator and pressure regulator/pressure regulator and others. The configurations of the pressure porting mechanisms include offset/offset, offset/hybrid functions, offset/linear, hybrid functions/hybrid functions, hybrid functions/linear.

The best assembly process for valve 100 and the alternative embodiments is accomplished as followings; for valve 100 (1) hinge boss 120 of leaflet 151 is compressed to a distance which is smaller than a gap between hinge bosses 120 (2) then leaflet 151 is inserted into body 101 from surface 109 with a rotary engagement between hole 165 and pin 118. For valve 100e (1) hinge boss 170e of leaflet 151e is compressed to a distance which is smaller than a gap between slots 117e 120 (2) then leaflet 151 is inserted into body 101e from surface 109e with a rotary engagement between boss 120e and slot 117e (3) spring 145e is expended to be disposed around pin 168e (4) two ends of one end of spring 145e is respectively disposed into a slot of pin 169e and slot 117e. For valve 100fg (1) leaflet 151g is inserted into port 106f (2) hinge boss 120f with spring 145f is through hinge hole 165f of leaflet 151f and threaded through body 101f from inside of body 101f (3) nut 197f is placed into hinge boss 120f to secure hinge boss 120f from outside of body 101f 170e (4) two ends of spring 145f are respectively disposed into a slot of pin 169f and slot 117f. (5) leaflet 151g is inserted into port 106f (6) hinge bosses 168 g is inserted into slot 115g (7) hinge boss 117g is lined up with 115g (8) shaft 195g is through holes 116g, 115g engaged with 167g with spring 145g (9) driver 196g is threaded into hole 116g and over top of spring 145g (10) two ends of spring 145g are respectively disposed into a slot of pin 169f and slot 117f (11) nut 197g is threaded into drive 196g to secure 196g (12) body 101f is threaded into body 101g.

The best assembly process for valve 200 and the alternative embodiments is accomplished as followings; for valve 200, for valve 100 (1) leaflet 251 is compressed and inserted to port 206 (2) slot 267 and slot 290 are respectively lined up with hinge boss 220 and boss 241. For 200h (1) two leaflet 251h are concentrically overlapped with hinge bosses 271h, 270h within two springs 245h in L block 296h (2) setscrew 246 is threaded into hole 240h of hinge boss 220h (3) Both of hinge boss 220h are placed on both top and bottom of the two leaflets 251 h by inserting pin 218h into hole 265h and spring 245h (4) assembled leaflets 251h are inserted into body 201h by matching up between slot 240h and boss 291h (4) setscrew 246h is screwed down into groove 211h to secure 220h.

The best assembly process for valve 300 and the alternative embodiments is accomplished as followings; for valve 300; (1) leaflet 351 is compressed and inserted to port (2) Boss 320 is lined up with slot 367. For 300b (1) spring 345b is disposed into slot 317b with engagement between section 347b and surface 324b, an end of section 346b and surface 323b ( 2) leaflet 351b is inserted in body 301b by disposing boss 370b into slot 317b

The best assembly process for valve 400 and the alternative embodiments is accomplished as followings; for valve 400 (1) boss 491 on leaflet 451 is compressed to a distance which is smaller than a gap between hinge bosses 420 (2) leaflet 151 is inserted into body 401 along with boss 420 until boss 491 pass boss 420.

In the best mode of operation, valve 100 and the alternative embodiments are the followings. With valve 100 installed in a native heart, when the heart starts to pump blood fluid into leaflet 151, because the upstream pressure is larger than that of downstream, the blood pressure difference between internal spherical surface 152 in the upstream and external surface 154 in the downstream generates a balanced force on center axial 110, but there is an offset between the pivot hinge axis and spherical shell axis, an opening torque is generated to actuate the leaflet 151 to an open position until surface 174 contacts surface 109. When the heart finishes the pumping cycle, the blood flow tends to flow back, the downstream pressure is larger than that of upstream, the pressure difference between two side surfaces and internal and external surfaces 152, 154 is reverse and generates a force and cause a closing torque until surface 173 contacts surface 109.

With 100b installed in a native heart, when the heart starts to pump blood fluid into leaflet 151b, because the upstream pressure is larger than that of downstream, the blood pressure difference on spherical shell section 158b does not generates a torque due to an zero distance between centric axis and pivot axis of section 158b, while actuation section 159b has an offset between a centric axis 161b and the pivot axis of section 158b, so the blood pressure difference generates a torque to open the valve 100b until hinge pin 122b full contact one of surfaces 172b. When the heart finishes the pumping cycle, the blood flow tends to flow back, the downstream pressure is larger than that of upstream, the pressure difference is reverse and generates a closing torque on section 159b and two side surfaces of leaflet 151b until hinge pin 122b full contact other surfaces 172b.

With valve 100hk used for a drainage system in medical applications, flow fluid from a heart comes into port 106g by means of surface 108g, because of a pressure difference between upstream of 108g and downstream of 109g and the hydride function mechanism in leaflet 151g, leaflet 151g as a check valve is rotated to an open position, then the flow get into port 106f and drains out through surface 109g , while valve 100k acts as a pressure relief valve to control the pressure difference between upstream and downstream, if the difference become too high to overcome a presetting value of spring 145g , the valve 100k will open to the atmosphere and reduce the difference.

With 100gh used for a chest drainage system in medical applications, flow fluid from the chest comes into port 106f by means of surface 106f, because of a pressure difference between upstream of 108f and downstream of 112g and offset mechanism in leaflet 151f, leaflet 151f as a check valve is rotated to an open position, then the flow get into port 106f and drains into outlet 112g, while leaflet 151g as a pressure regulator control the pressure difference upstream and downstream, if the difference become too high to overcome a presetting value of spring 145g, the leaflet 151g will open to the atmosphere and reduce the difference.

With valve 100g for a two-way check system with two inlets and one outlet, flow A comes into port 106g by means of surface 108g, flow B comes into hole 112g, flow A or B flow out by means of surface 109g on port 106g. When a pressure difference between upstream of 112g and downstream of 109g increase to overcome a torque provided by spring 145g, leaflet 151g is rotated to an open position and cover hole 112g, so the flow A get into port 106f and drains out through surface 109g . When a pressure difference between upstream of 112g and downstream of 109g decreases, a torque provided by spring 145g is to move the leaflet 151g to a closed position, hole 112g is uncovered, so the flow B get into port 106f and drains out through surface 109g on port 106g.

In the best mode of operation, valve 200 and the alternative embodiments, are the followings. With valve 200 installed in a native heart, when the heart starts to pump blood fluid into internal surface of leaflet 251, because the upstream pressure is larger than that of downstream, the blood pressure difference between internal spherical surface 252 and external surface on section 258 generates a force in a centric axis of leaflet 251, but no torque is generated due to zero offset between centric axis and pivot axis, while the difference on actuation section 259 generates an force on centric axis and torque due to an offset between the centric axis and pivot axis on the actuation section 259. When the heart finishes the pumping cycle, the blood flow tend to flow back, the downstream pressure is larger than that of upstream, the pressure difference is reverse and generates a closing torque on section 259 and two side surfaces of leaflet 251 until side surfaces of leaflet 251 contact each other then slot 290 contacts lock slot 241.

With valve 200a installed in a native heart, when the heart starts to pump blood fluid into internal surface of leaflet 251a, because the upstream pressure is larger than that of downstream, the blood pressure difference between internal spherical surface 252a and external surface on section 258a generates a force in a centric axis of leaflet 251 a, but no torque is generated due to zero offset between the centric axis and the pivot axis, while the difference on cylindrical actuation section 259a generates an force on centric axis and torque due to an offset between the centric axis and pivot axis on the actuation section 259a. When the heart finishes the pumping cycle, the blood flow tend to flow back, the downstream pressure is larger than upstream, the pressure difference is reverse and generates a closing torque on section 259a and two side surfaces of leaflet 251a until side surfaces of leaflet 251b contact each other then slot 272a contacts lock boss 222a.

With valve 200h installed in a pipe line, when a pressurized fluid flows into internal surface of leaflet 251h, because the upstream pressure is larger than that of downstream, the fluid pressure difference between internal spherical surface 252h and external surface on section 258h generates a force in a centric axis of leaflet 251, but no torque is generated due to zero offset between the centric axis and the pivot axis, while the difference on the actuation section 259h generates an force on centric axis and torque due to an offset between the centric axis and pivot axis on the actuation section 259h to overcome torque on spring 245h until leaflet 251h contact surface 223h. When flow finishes the pumping cycle, the flow tend to flow back, the downstream pressure is larger than that of upstream, the pressure difference on section 259h and two side surfaces of leaflet 251h is reverse, leaflet 251h with the tied spring 245h generates a closing torque until side surfaces of leaflet 251h contact each other then slot 272h contacts lock boss 222h.

In the best mode of operation, valve 300 and the alternative embodiments are the followings. With valve 300 installed in a native heart, when the heart starts to pump blood fluid into internal surface of leaflet 351, because the upstream pressure is larger than that of downstream, the blood pressure difference between internal spherical surface 352 and external surface on section 358 generates a force in a centric axis of leaflet 351, but no torque is generated due to zero offset between centric axis and pivot axis, while the pressure difference on actuation section 359 generates an force on centric axis and torque due to an offset between the centric axis and pivot axis on the actuation section 359 until surface 374 contacts surface 324. When the heart finishes the pumping cycle, the blood flow tend to flow back, the downstream pressure is larger than that of upstream, the pressure difference is reverse and generates a closing torque on section 359 and two side surfaces of leaflet 351 until side surfaces of leaflet 251 contact each other then surface 373 contacts lock slot 323.

In the best mode of operation, valve 400 and the alternative embodiments are the followings. With valve 400 installed in a native heart, when the heart starts to pump blood fluid into internal surface of leaflet 451, because the upstream pressure is larger than that of downstream, the blood pressure difference between internal spherical surface 452 and external surface on section 458 generates a force in a centric axis of leaflet 451, but no torque is generated due to zero offset between centric axis and pivot axis, while the pressure difference on actuation section 459 generates an force on centric axis and torque due to an offset between the centric axis and pivot axis on the actuation section 459 until slot 472 contacts stopper 422. When the heart finishes the pumping cycle, the blood flow tend to flow back, the downstream pressure is larger than that of upstream, the pressure difference is reverse and generates a closing torque on section 459 and two side surfaces of leaflet 451 until lock boss 491 hits lock slot 441, then lock boss is compressed to a side due to a release slot 495, then side surfaces of leaflet 451 contact each and provides a seal.

The present invention first developed a mechanical heart valve which mostly resembles to human heart valve in the performances over all existing mechanical heart valves.

(1) One constant central flow stream.

This invention is provided with mechanical heart valves 100,200,300,400 and the alternative embodiments having a constant spherical engagement and seal between the body and the. Those heart valves have a cyclical opening for maximum of flow area and keep one constant central flow stream when the valves are approached to full closed position or open position, as a result pressure loss reduce, so does the blood cell damage.

(2) No obstructive in the center flow stream.

This invention provides mechanical heart valves 100,200,300,400 and the alternative embodiments with a constant spherical engagement and seal between the body and the leaflet, those heart valves not only provide no obstructive in the center flow stream, but also retreat the leaflet or the leaflets to the body side wall and reduce the contact area with blood flow stream, as a result pressure loss reduce, so does the blood cell damage.

(3) No closed force between valve body and valve leaflet

This invention provides mechanical heart valves 100,200,300,400 and the alternative embodiments with a constant spherical engagement and seal between the body and the leaflet, so those valves are closed with radial seals between the body and leaflet without high closed forces unlike the conventional hear valve with high impact force on the face to face seal between the valve body and leaflets. According to the medical researches, on an average, the mechanical heart valve in an adult life patient operates about 200 million times, since no material like human heart valve muscle is developed for the mechanical heart valve, every closed operation of the 200 million time does damage the blood cells and the valve one way or other. With this invention, 200 million time of the operation is eliminated while countless blood cells are saved, and the mechanical valve can be used for much longer time without damage.

(4) Soft closing between the leaflet.

This invention provides an unique solution to the problem the conventional mechanical heart valves are faced. With this two prone approaches, first is to provide proper and adequate closing torque and energy by designing a proper offset distance for eccentric actuation mechanism or proper actuation section for the hybrid function mechanism, so no excessive energy is employed to close the leaflet as indicated leaflets 151,251,351,451, second is to have a soft closing process, this process is provided with a dumping mechanism to dissipate the kinetic energy of the leaflets 151,251,351,451, with more dry dumping with valve body 101,201,301,401 and leaflets 151,251,351,451 and less viscous damping between the valve 100,200,300,400 and blood fluid. It is unlike the conventional mechanical heart valve which has most damping effect between blood and the leaflet and the valve body, such method cause more blood cell damage. The soft closing process comprise two stages; (a) impact/lock (b) contact/seal, as valve 100 shows with the eccentric cam mechanism, at the first stage that the leaflet 251 is partially engaged with body 201, the second stage is that the leaflet 251 is fully engaged with body 201, the block is provided with additional dumping effect, minimize the cavitations when fully engaged with the with body 201 as valve 100 shows with the eccentric cam mechanism, at the first stage that the leaflet 251 is partially engaged with body 201, the second stage is that the leaflet 251 is fully engaged with body 201, the block is provided with additional dumping effect, minimize the cavitations when fully engaged with the with body 201

(5) Self absorb clot material

In fact, human heart valve is a live organ, the lifeless mechanical heart valve never has such a self absorb ability, even with pyrolitic carbon material, the coagulation is inevitable for a lifeless device, however, to a certain extent, the mechanical heart valve can reach a closed result to reduce the clot accumulation, but the most important thing is to reduce the root cause of clot, blood cell damage at first place. The present invention provides a long sought, comprehensive solution to the problem. This solution include two steps, first is to reduce the damage of blood cell with each feather in this invention, second is to provide mechanical and medical cleanup methods. By design, the leaflet 151,251,351,451 along with bodies 101,201,301,401 constitute cutters, whenever the leaflets open or closes, the leaflets clean up the porting surfaces, if any clot is established, the relative movement between leaflet and the body will remove the clot, while the medical method is to coated with anti clot drug on the leaflet external surface and the body internal surfaces, so rubbing between the two surfaces will release the drug gradually. So the fundamental difference between this invention and prior arts is with this two prone approaches. so the process of rubbing between balls 146 and rotor 152 or body 153 is a process of drug releasing, such approaches are complete different from the conventional methods which are eight to prevent the coagulation which is impossible, or to ask the patients to take the anticoagulant drug for life which is troublesome, but here this mechanical heart valve is based on the assumption that the coagulation is inevitable, this mechanical heart valve is an anticoagulant drug and cleanup device for life.

(6) Leaflets internal surface contact flow stream,

As we know any contained fluid will form a boundary layer which contact solid wall, the boundary layer has zero velocity, so the velocity difference cause blood cell damage and pressure loss, more contact area between valve and blood fluid, more blood cell damage and more pressure loss. This invention mover a step further, in fact, the contact area at open position is smaller than the contact area in closed position, the leaflets 151,251,351,451 retreat the most part and cover most of internal surfaces of bodies 101,201,301,401, even better than human heart valve in terms of contact area.

(7) Valve body and leaflet as a integral part

Why we need mention this, because the nature of the human heart valve with the body and leaflet as a integral part dramatically improves the dynamitic performance of the heart valve, first there are no leakage and closing force between the valve body and leaflet, second most importantly, when the valve is closed, the impact energy among the leaflets is absorb by entire valve body or heart unlike conventional mechanical heart valve only the leaflet absorb the impact energy and it take much long time to reach stable condition. But with the impact/lock and contact/seal mechanism in this invention, mechanical heart valve can have the same performance as human heart valve, when the leaflet is approached to a close position, the lock boss and lock slot impact and lock together either with each other or with valve body, as we can find as the mass increase, the impact energy effect decrease, even the oscillation still exist for a while, because the leaflets lock with each other, there is no leakage or the cavitations.

(8) Seal only among the leaflets.

This invention provide a face to face seal for bi or triple leaflets mechanical heart valve and radial seal between the valve body and leaflets and no leaflet seal for one leaflet, all those seals has a similar sealibilty as the human heart valve does, especially the one leaflet mechanical heart valve has a better sealibilty.

(9) Shorter travel between open and closed positions

This invention provides one, two and three leaflet structures. In case of smaller size valve, the benefit with the triple leaflets structure is insignificant, in case of large size valve, this invention provide a reliable and simple triple leaflet mechanical heart valve.

(10) No hinge mechanism

The human valve has no hinge mechanism and eliminate all disadvantages of the hinge mechanism, such as fluid stagnation, hinge wearing out, blood cell damage, eccentricity which cause poor dynamic performance of the leaflet, but the hinge mechanism provide a simple rotation function. This invention provides mechanical heart valve with various improved hinge mechanisms to minimize the disadvantages of conventional hinge mechanisms. First the hinge mechanisms in valves 100,100a,100b 100d,100f,100h ,200,200a ,200c,200d ,200e,300300a, 400,400a are located on outward surfaces or as projection forms on the valve bodies to eliminate or reduce the fluid stagnation and blood cell damage. Second the full or partially cylindrical pin and hole engagements in 100, 100a,100b,100c,100d, 200,200a ,200b ,200c, 200d ,200e, 400,400a improve the concentricity and the dynamic performance of leaflet and reduce the hinge wearing. Third one hinge pivot axis feature in valves 100,100a,100b 100d,100f,100h,200d are provided with the highest dynamic performance and synchronization of the leaflet.

(11) Longevity

The invention provide the mechanical heart valve with a best fluid dynamics and optimized valve structure with less closing impact forces, so the life expediency can last about 20-40 years which is enough for most adult patients with only operation and quality life after the implanted

In terms of the weakness of human heart valve
(1) Center jet flow leakage when the three leaflets are closed

This invention provides better structures to overcome the problem, no mechanical heart valves in this invention has this problem.
(2) Higher energy consumption to rotate the leaflet with the large normal surface of leaflet to the flow stream.

This invention provides the better fluid dynamic structure with the spherical shell leaflet having a small cross section which is normal to flow stream, it takes about ⅕ energy that human heart valve consumes.
(3) Susceptible to disease

This invention provides a better materials than human heart valve tissue in terms of resistance to diseases.

The present invention provides two novel rotary pressure porting mechanisms which are behind the mechanical heart valve in this invention. If the first successful mechanical heart valve with a caged ball valve, pioneered by Starr and Edwards was inspired by Williams's ball valve on U.S. Pat. No. 19,323 (1858), then this invention is an opposite example, the rotary pressure porting mechanisms on the mechanical heart valve are inspired by the process to mimic our human heart valve. For more then 100 years, the liner pressure porting mechanism plays a key role in our life as William did, but it's main disadvantage is obstruction of central flow stream, while the conventional rotary pressure porting mechanism with conventional hinge device came out with improvement of center flow stream, but it still blocks flow stream and is a size dependent mechanism, as size of the valves increase, so dose the torque to operate the valves, finally the actuation mechanism of the valve member is based on two opposite actuation sections, only the difference area on the two sections play a actuation role, as a result, the actuation mechanism wastes the space of the valve member and actuation energy. But those rotary pressure porting mechanisms in invention show three main advantages over prior arts beside the advantages mentioned in the mechanical heart valve as followings

(1) A design dependant mechanism. With the offset spherical mechanism, the operating torque is depend on the diameter of spherical the valve member, flow pressure and the offset between the center axis and the pivot axis, while the hybrid function mechanism is depend on the flow pressure and the area of actuation section of the valve member, so as the sizes of flow pipe or tubing increase, the size of valves based on those two pressure porting mechanisms are not necessary to increase, the offset or the selected actuation area can be decisive factors, as a result, the energy to actuate the valve members 151,251,351,451 is about ⅕ of the conventional mechanical heart valves consume (2) optimized actuation torque. The conventional pressure porting mechanisms have no way to control closing force or torque, so in most cases, the closing force either is so strong and damage the valve member or so weak and cause chatter With this invention, the closing force can be designed to close the valve member fast enough but not so strong that damage the valve member and cause chatter.(3) a quiet and soft closing. With valves 100, there is no slump closing while with 200, there is impact/lock and contact/seal mechanism, those features play a key in the fuel delivery systems, especially in liquid oxygen and hydrogen applications, any hard closing in a check valve, pressure relief valve or pressure regulator can case explosion, a real soft or no slum closing is critical requirement for such system, or medical devices used in a hospital environment, quiet closing is very important for the patient as well as doctors finally a quiet soft closing can prolong the valve life and reduce the noise and vibration. (4) Versatility. Above all those two rotary pressure porting mechanisms can be applied to a check valve or pressure relief valve or pressure regulator or the combination like valves 100f, 100g, those systems are much compact, reliable and efficient and are used in many fields like medical devices, commercial building or housing, HVAC, industrial process, food process, chemical plant oil and refinery, automotive, aerospace machine tools.

The present invention provides other long sought solution to the backflow problem on the mechanical heart valve. The native heart valve has also backflow problem, but the consequence of backflow is not as serious as in the mechanical heart valve, the difference is that a live tissue which has muscles and self repair functions while the mechanical heart valve is a lifeless device without muscles and self repair functions, so this valve is provided with backflow preventing system with three solutions, mechanical solution with watch spring 192 and magnetic for the highest reliability, the magnetic and mechanical solution with blocks 140 and 170 is the choice, for patient who has a pace maker, the mechanical solution is the choice with safety closure, so there is no possibility that the spring 192 falls into downstream.

The present invention provides various novel hinge mechanisms for different applications. The followings are the main advantages

(1) Simplicity. Most hinges mechanisms in this invention are provided with simple profiles or shape, cylindrical or flat or the combination on the hinge pins or hinge holes or the boss or slot.

(2) Less stagnation. The hinge mechanisms in valves 100,100a,100b 100d,100f,100h,200,200a,200c,200d,200e,300300a, 400,400a are located on outward surfaces or as projection forms on the valve bodies to eliminate or reduce the fluid stagnation and blood cell damage.

(3) High concentricity. The valves 100, 100a, 100b, 100c, 100d, 200,200a, 200b,200c, 200d ,200e ,400,400a are provided with the full or partially cylindrical pin and hole engagements to improve concentricity, synchronism of leaflets and the dynamic performance of leaflet and reduce the hinge wearing, vibration and noise., more importantly one hinge pivot axis features in valves 100,100a,100b 100d,100f,100h, 200d are provided with the highest dynamic performance and synchronization of the leaflets.

(4) Versatile. Most hinge mechanisms in this inventions are provided with other functions such as stopper, locking devices and backflow preventing devices, so the valve can be designed to much compact.

(5) Reliability

The reliable design features are presented through the entire invention. (a) Less moving part or any part. With one moving leaflet 151 in valve 100 and the alternative embodiments, the valves are more reliable than any mechanical heart valve with two or more part or moving parts (b)redundancy. With the dual redundant hinge mechanisms, beside hinge pin/ hinge slot, the spherical surface in the body/spherical surface in the leaflet is provided as a secondary rotation mechanism, so in case of wearing out of the hinge mechanism, the leaflet is still workable (c) inclusive designs. An inclusive designs includes a falling out proof and a loosing proof. The springs in 100h,100f,200g,200f,200h have losing proof design, while the setscrew in valve 200h and the spring in 300b have the falling proof design, in case of the screw or spring breaking down all those parts will not fall into the flow stream. The inclusive designs also can eliminate leakage path like the hinge device in valve 200h without any hinge hole through the body.

I claim:

1. A multiple pressure porting means for regulating fluid comprising;
(a) A first valve having a valve body including at least one fluid passageway defined by at least one internal spherical surface and a shell valve member movably disposed in said body by a hinge means having a spherical balance section engaged with said internal spherical surface and an actuation section for generating rotary movement under fluid pressure difference between an inward surface of said actuation section and an outward surface of said actuation section
(b) A second valve connected with said first check valve having a body including at least one fluid passageway defined by at least one internal spherical surface with one fixed centric axis and one valve member movably disposed in said body by a hinge means having a spherical shell with an eccentric pivot axis, a distance between said centric axis and said eccentric pivot for generating rotary movement under fluid pressure difference between an inward surface of said valve member and an outward surface of said valve member

2. The multiple pressure porting means of claim 1, further including a pressure relief valve connected with said second valve for a drainage system.

3. A rotary pressure porting means for regulating fluid comprising;
(a) A valve body having at least a fluid passageway defined by at least one internal spherical surface with a fixed centric axis;
(b) At least a valve member movable disposed in said body having a pivot axis which is concentric with said fixed centric axis, said valve member having a spherical shell balance section engaged with said internal, spherical surface and an actuation section for generating rotary movement under fluid pressure difference between an inward surface of said actuation section and an outward surface of said actuation section, a profile diameter of said internal spherical of said body is substantially same as an external profile diameter of said spherical shell balance section of said valve member;
(c) A hinge means between said body and said valve member for controlling a rotary movement of said valve member;

4. The pressure porting means of claim 3, wherein said hinge means comprises a pair of first hinge bosses defined by said fixed axis, each of said first bosses having a concentric pin and a pair of second hinge bosses defined by said pivot axis of said valve member, each of said second bosses having a hinge hole receiving said pin with a clearance, said hinge means further include a pair stopping means having a pair of partial, cylindrical shell stopper located at top and bottom of said valve member, each of said stoppers has two stopping surfaces to prevent said valve member over travel at both closed and open positions.

5. The pressure porting means of claim 3, wherein said hinge means comprise a pair of first hinge bosses defined by said fixed axis, each of said first bosses having a concentric, S shape hinge slot defined by two concentric cylindrical surfaces and two pair of stopping surfaces and a pair of second bosses on said valve member movable disposed in said body defined said pivot axis and two concentric cylindrical surfaces and two pair of stopping surfaces. Said stopping surfaces on first bosses are against said stopping surfaces on said second bosses when valve member said are at both closed and open positions.

6. The pressure porting means of claim 3, wherein said hinge means comprise a pair of first hinge pins defined by said fixed axis located at top and bottom of said fluid passageway, each of said pins having an eccentric cylindrical stopper defined by an eccentric, fixed axis and a pair of hinge holes located on top and bottom of said valve member, each of said hinge holes having a pair of equally eccentric stopping cylindrical surfaces on opposite side, a distance between said hinge pin and said eccentric stopper on said body is substantially same as that of between said hinge hole and said eccentric stopping surfaces on said valve member. Said stopping surfaces on said hinge holes are against said stopping surfaces on said stopper when said valve member are at both closed and open positions.

7. The pressure porting means of claim 3, wherein said hinge means comprise a pair of hinge slot located on top and bottom of said body defined by a centric fixed axis, two concentric cylindrical surfaces and two stopping surfaces and a pair of bosses on said valve member movable disposed in said hinge slot defined said pivot axis and two concentric cylindrical surfaces and two pair of stopping surfaces. Said stopping surfaces on said bosses are against said stopping surfaces on said hinge slot when valve member said are at both closed and open positions.

8. The pressure porting means of claim 3, wherein said hinge means is located on a top front area and a bottom back area of said valve.

9. The pressure porting means of claim 3, wherein said hinge means comprise a pair of first hinge pins defined by said fixed axis located at top and bottom of said fluid passageway, each of said pins having an eccentric cylindrical stopper defined by an eccentric, fixed axis and a pair of hinge holes located on top and bottom of said valve member, each of said hinge holes having a pair of equally eccentric stopping cylindrical surfaces on opposite side, a distance between said hinge pin and said eccentric stopper on said body is substantially same as that of between said hinge hole and said eccentric stopping surfaces on said valve member. Said stopping surfaces on said hinge holes are against said stopping surfaces on said stopper when said valve member are at both closed and open positions.

10. A pressure porting means for regulating fluid comprising;
(a) A body including one fluid passageway defined by at least one internal spherical surface having one fixed centric axis
(b) At lease a valve member movably disposed in said body having a spherical shell with an eccentric pivot axis, a distance between said centric axis and said eccentric pivot for generating rotary movement under fluid pressure difference between an inward surface of said valve member and an outward surface of said valve member, a profile diameter of said internal spherical surface of said body is substantially same as an outside profile diameter of said spherical shell
(c) A hinge means between said body and at lease one said valve member for controlling said valve member movement

11. The pressure porting means of claim 3, wherein said hinge means for two valve members comprises four pivot hinge bosses and two lock bosses equally located on top and bottom of said fluid passageway, each of said pivot bosses defined by said fixed axis and two concentric cylindrical surfaces and two flat surfaces, each of said lock bosses defined by two eccentric cylindrical surfaces and two hinge slots and two lock slots equally located on top and bottom of each of said two valve members, having a concentric pin and a pair of second hinge bosses defined by said pivot axis of said valve member, each of said second bosses having a hinge hole receiving said pin with a clearance, said hinge means further include a pair stopping means having a pair of partial, cylindrical shell stopper located at top and bottom of said valve member, each of said stoppers has two stopping surfaces to prevent said valve member over travel at both closed and open positions.

12. The pressure porting means of claim 3, wherein said hinge means for two valve members comprises four hinge slots equally located in an opposite direction inside of said body and one hinge boss with a lock boss, one hinge boss with a lock slot in an opposite direction on said valve member. Each of said hinge slots is defined by two cylindrical surfaces, two stopping surfaces, said hinge boss movably engaged with said hinge slot is defined by two cylindrical surface and two flat surfaces. A diameter of said lock slot is slightly larger than that of said hinge boss, but an opening of said lock slot is slightly smaller than that of said lock boss, so when said valve member is approached to a closed position, said lock boss first impacts said lock slot, a snap action takes place, then said valve member contact either other and provide a seal, said stopping surfaces is provided to prevent said valve member to over-travel.

13. The pressure porting means of claim 3, wherein said hinge means for two valve members comprises two fixed hinge bosses equally located on top and bottom of said fluid passageway and two pivot hinge bosses in an opposite direction on said valve member, each of said fixed bosses is defined by one outward cylindrical surface, one inward cylindrical surfaces connected two stopping surfaces, a top of said two pivot bosses has a hinge hole to movably engaged with said outward surface of fixed bosses, a bottom of said two pivot bosses has a hinge slot defined by one outward cylindrical surfaces, one inward cylindrical surfaces and two stopping surfaces engaged with said fixed boss, said two stopping surfaces on said fixed bosses are against said two stopping surfaces on said hinge slot of said pivot bosses when said valve member is at both open and closed positions for preventing a over-travel of said valve member.

14. The pressure porting means of claim 3, wherein said hinge means for said two valve members comprises two cylindrical hinge slots on said fluid passageway in an opposite direction and two partially cylindrical hinge bosses with two setscrews respectively disposed on said hinge slots and two pivot hinge bosses on said valve member, each of said hinge slots has a concentric, conical groove to receive one conical end of said setscrew and a lock cylindrical slot to receive a hinge pin of said hinge block for preventing said hinge block falling out. Said hinge boss has also a hinge pin to hold said valve members in an overlapping manner and a bottom larger hole extended to a smaller thread hole engaged with one threaded end of said setscrew.

15. The pressure porting means of claim 10, wherein said hinge means for said two valve members comprises two cylindrical hinge slots on said fluid passageway in an opposite direction and two partially cylindrical hinge bosses with two setscrews respectively disposed on said hinge slots and two pivot hinge bosses on said valve member, each of said hinge slots has a concentric, conical groove to receive one conical end of said setscrew and a lock cylindrical slot to receive a hinge pin of said hinge block for preventing said hinge block falling out. Said hinge boss has also a hinge pin to hold said valve members in an overlapping manner and a bottom larger hole extended to a smaller thread hole engaged with one threaded end of said setscrew.

16. The pressure porting means of claim 3, wherein said hinge means for two valve members comprises four hinge slots on said fluid passageway in an opposite direction, two hinge bosses on said valve member in an opposite direction and four L springs disposed between said hinge slots and said hinge bosses, each of said hinge slots is defined by a pair of cylindrical surfaces and two pair of two surfaces, each of said hinge bosses movably engaged with said hinge slot is defined by two cylindrical surfaces and two surfaces, said four L springs are respectively disposed in said hinge slots, a closing torque is provided by one end of said L spring biased against said surface of said hinge boss and other end of spring biased against said flat surface. When said valve member is approached to open position, said hinge boss is moving against said end of spring until spring to contact surface. When said valve member is approached to a closed position, said valve member is actuated by both flow and said spring.

17. The pressure porting means of claim 3, wherein said hinge means for said three valve members comprises three hinge bosses, three stoppers equally spanned on said fluid passageway of said valve body and two lock bosses, two release slots, two hinge slots on said valve member in an opposite direction, said two hinge slots are respectively movably engaged with said hinge boss member. Each of said hinge bosses is defined by a top spherical internal surface, two side cylindrical surfaces, two flat surfaces and two lock corners, a profile diameter of said hinge slot is substantially the same as that of said side cylindrical surface of said hinge boss, said hinge means also comprises a stopper slot on said valve member being against said spherical boss to prevent any over-travel of said valve member when said valve member is at an open position, when said valve member is approached to a closed position, a first of said lock bosses impacts said lock corner, a second of said lock bosses is further compressed to a side and locked with said corner, then said side surfaces of said valve members contact each other and provide a seal.

18. The pressure porting means of claim 3, wherein said hinge means for said three valve members comprises three bottom hinge bosses, three top hinge bosses three stoppers equally spanned on said fluid passageway of said valve body and two bottom hinge slots movably engaged respectively with said bottom hinge bosses and two top hinge slots movably engaged respectively with said top hinge bosses, two lock bosses, two release slots on said valve member in an opposite direction. Each of said top hinge bosses is defined by a top spherical surface, two cylindrical surfaces Each of said bottom hinge bosses is concentric with top hinge boss and is defined by a top spherical internal surface and two side cylindrical surfaces and two surfaces and lock corners. a profile diameter of said top hinge slot is substantially the same as that of said side cylindrical surface of said top hinge boss, said hinge means also comprises a stopper slot on said valve member being against said stopper to prevent any over-travel of said valve member when said valve member is at an open position, when said valve member is approached to a closed position, a first of said lock bosses impacts said lock corner, a second of said lock bosses is further compressed to a side and locked with said corner, then said side surfaces of said valve members contact each other and provide a seal.
